SEQUENCE LISTING (2 sequences)

SEQ ID NO: 1 - DNA, 50 bp, Unknown
  V3 and V4 regions of 16S gene forward PCR primer
  misc_feature (42)..(42): n is a, c, g, or t
tcgtcggcagcgtcagatgtgtataagagacagcctacgggnggcwgcag

SEQ ID NO: 2 - DNA, 55 bp, Unknown
  V3 and V4 regions of 16S gene reverse PCR primer
gtctcgtgggctcggagatgtgtataagagacaggactachvgggtatctaatcc

(19) United States
(12) Patent Application Publication
(10) Pub. No.: US 2020/0405787 A1
(43) Pub. Date: Dec. 31, 2020

(54) NEW USE OF MICROBIOLOGICAL COMPOSITIONS

(71) Applicant: Adare Pharmaceuticals SAS, Houdan (FR)

(72) Inventors: Colin HILL, Friars Walk, Cork (IE); Ted DINAN, Carrignafoy, Cobh, Cork (IE); Alicja WARDA, Lavitt's Quay, Cork (IE); Stephen PERRETT, Princeton, NJ (US)

(21) Appl. No.: 16/981,737

(22) PCT Filed: Mar. 22, 2019
(86) PCT No.: PCT/EP2019/057346

§ 371 Date: Sep. 17, 2020

Related U.S. Application Data
(60) Provisional application No. 62/646,570, filed on Mar. 22, 2018.

(51) Int. Cl. A61K 35/747; A61P 25/34; A61K 9/19; A61K 47/26; A61P 25/22; A61P 25/24; A23L 33/135

(52) U.S. Cl. CPC A61K 35/747; A61P 25/34; A61K 9/19; A61K 47/26; A23Y 2220/29; A61P 25/24; A23L 33/135; A23V 2002/00; A23Y 2220/35; A61P 25/22

(57) ABSTRACT

The present invention provides a pharmaceutical composition comprising dead cells of Lactobacillus strains useful for the protection of subjects against the development of conditions with behavioral, psychological and/or physical components caused or exacerbated by stress or anxiety, and/or useful in treating existing conditions with behavioral, psychological and/or physical components caused or exacerbated by stress or anxiety. Examples of specific conditions include stress, anxiety, depression, mood disturbances, sociability disorders, irritable bowel syndrome, autism, autism spectrum disorder, post-traumatic stress disorder, chronic stress and a range of other stress-related diseases.

### BACKGROUND OF THE INVENTION

The present invention relates to microbiological compositions having psycholeptic (calming) and psychoanaleptic (stimulating) effects, which can be used to protect healthy subjects against the development of conditions with behavioral, psychological and/or physical components caused or exacerbated by stress or anxiety, and/or positively impact existing conditions with behavioral, psychological and/or physical components caused or exacerbated by stress or anxiety.

The intestinal microbiota is increasingly being recognized as a major modulator of the central nervous system (CNS), establishing a significant field of scientific study which has been termed ‘the microbiota-gut-brain axis’, a bi-directional communication system comprising neural connections, endocrine and immune signaling. Convincing evidence exists for a role of the gut microbiota composition in the regulation of the key stress hormones cortisol (in humans) and corticosterone (in mice), with similar hormone analogues in other species. Gut microbiota interventions, including probiotic and prebiotic use, have resulted in positive effects in some cognitive and behavioral conditions in animals, including humans.

The gut microbiota has principally been exploited to yield positive effects on brain health via probiotics, with various Bifidobacterium and Lactobacillus strains shown to have anxiolytic and pro-cognitive effects in both rodents and humans. In further reports, e.g. International published patent application numbers WO 2016/069795 and WO 2014/036182, probiotics such as Bacteroides bacteria have demonstrated positive effects on autism spectrum disorders. However, although single or multi-strain probiotics have the potential to modify cognitive function and/or behavior, their tendency to provide relatively narrow spectrum effects on the microbiome is a limitation. Live bio-therapeutics also have significant drawbacks when used as therapeutic agents. Gastric acid, the digestive process and anaerobic conditions in the colon are formidable obstacles to the viability of live bio-therapeutics in the distal gut. Furthermore, live bio-therapeutics are generally administered as many billions of colony-forming units, and in immune-compromised subjects, the very young and very old, live bio-therapeutics can become an infection hazard. Live bio-therapeutics also have the potential to transfer drug-resistance or bacterial-virulence gene cassettes. Additionally, live bio-therapeutics are difficult products to stabilize and standardize, and ‘chemistry, manufacturing and controls’ are significant challenges. To date, no live bio-therapeutic has been approved as a pharmaceutical product in modern, well-regulated markets.

Lactobacillus is a genus of gram-positive, facultative anaerobic or microaerophilic, rod-shaped, non-spore-forming bacteria. They are a major part of the lactic acid bacteria group (i.e. they convert sugars to lactic acid). In humans, they constitute a significant component of the microbiota at a number of body sites. Lactobacillus currently contains over 180 species and encompasses a wide variety of organisms.

We have now surprisingly found that compositions containing heat-killed cells of specific strains of Lactobacillus, and, in particular, when such cells are in combination with culture medium, have psycholeptic and psychoanaleptic effects. These compositions are particularly useful for treating stress or anxiety, and/or protecting against conditions with behavioral, psychological and/or physical components caused or exacerbated by stress or anxiety, including, for example, depression, mood disturbances, conditions where sociability is dysfunctional, autism, autism spectrum disorder, post-traumatic stress disorder, chronic stress and a range of other stress-related diseases, and irritable bowel syndrome. These compositions may also be useful to protect subjects against the effects of fibromyalgia, obsessive-compulsive behavior, addiction or addictive behavior and its treatment. Compositions containing heat-killed cells of specific strains of Lactobacillus, and, in particular, when such cells are in combination with culture medium, are particularly effective in protecting healthy subjects against conditions caused or exacerbated by elevated stress and/or anxiety levels.

Without wishing to be bound to any one mechanistic theory, heat-inactivated bacteria of the present invention also have profound effects on the gut microbiota, with changes in both composition and diversity. It is therefore postulated that the bacteria are psychobiotics, i.e. they are exhibiting a bacterially-mediated influence on the brain.

One particular product of the present invention is Lacteol®. Lacteol® is sold as a symptomatic treatment for diarrhea in adults and children supplemental to rehydration and/or dietary measures. However, it has not to date been reported to exhibit psycholeptic or psychoanaleptic effects, or to have any influence on the microbiota-gut-brain axis.

The active component in Lacteol® is derived from a culture solution containing heat-killed cells of Lactobacillus LB strain (a combination of Lactobacillus fermentum, Lactobacillus delbrueckii) and fermented culture medium. Lacteol®, along with other active products of this invention comprising dead Lactobacillus cells, have a number of potential advantages over products containing live organisms, such as probiotics, when used to prevent or positively impact conditions with behavioral, psychological and/or physical components caused or exacerbated by stress or anxiety. Particular advantages of using dead organisms include consistency of composition and effect, ease of storage, no risk of infection in vulnerable patients, no translocation of bacterial-virulence or antibiotic-resistance cassettes, and the product retains activity when used in conjunction with antibiotics or anti-fungal agents.

### SUMMARY OF THE INVENTION

Throughout this document the terms “treatment” and “treating” are intended to also cover the preventative and protective uses of a composition of the present disclosure against a stated condition or disorder.

One aspect of the present disclosure provides a composition comprising dead cells of Lactobacillus fermentum and/or dead cells of Lactobacillus delbrueckii for use to produce a psychobiotic effect in a human or non-human animal subject.

Another aspect provides a composition comprising dead cells of Lactobacillus fermentum and/or dead cells of Lactobacillus delbrueckii, for use to produce a psychobiotic effect in a human or non-human animal subject, wherein said psychobiotic effect is achieved by changing the composition and/or diversity of the human or non-human animal gut microbiota.

A further aspect provides a composition comprising dead cells of Lactobacillus fermentum and/or dead cells of Lactobacillus delbrueckii, for use to produce a psychobiotic effect in a human or non-human animal subject, wherein said psychobiotic effect is achieved by modifying (e.g. reducing) the amount of Alistipes and/or Odoribacter species present in the human or non-human animal gut.

Another aspect provides a method of treating conditions with behavioral, psychological and/or physical components caused or exacerbated by stress or anxiety in an animal (including human) patient, comprising administering to said patient a psycholeptic and/or psychoanaleptic effective amount of dead cells of Lactobacillus fermentum and/or dead cells of Lactobacillus delbrueckii, including dead cells of Lactobacillus LB.

A further aspect provides a method of treating an animal (including human) patient with mood disturbances caused or exacerbated by stress or anxiety, comprising administering to the patient an effective amount of dead cells of Lactobacillus fermentum and/or dead cells of Lactobacillus delbrueckii, including dead cells of Lactobacillus LB.

A further aspect provides a method of treating an animal (including human) patient with autism or autism spectrum disorder (ASD) exacerbated by stress or anxiety, comprising administering to the patient an effective amount of dead cells of Lactobacillus fermentum and/or dead cells of Lactobacillus delbrueckii, including dead cells of Lactobacillus LB.

A further aspect provides a method of treating an animal (including human) patient with stress or anxiety, comprising administering to the patient an effective amount of dead cells of Lactobacillus fermentum and/or dead cells of Lactobacillus delbrueckii, including dead cells of Lactobacillus LB.

A further aspect provides a method of treating an animal (including human) patient with a sociability disorder caused or exacerbated by stress or anxiety, comprising administering to the patient an effective amount of dead cells of Lactobacillus fermentum and/or dead cells of Lactobacillus delbrueckii, including dead cells of Lactobacillus LB.

A further aspect provides a method of treating an animal (including human) patient with depression caused or exacerbated by stress or anxiety, comprising administering to the patient an effective amount of dead cells of Lactobacillus fermentum and/or dead cells of Lactobacillus delbrueckii, including dead cells of Lactobacillus LB.

A further aspect provides a method of treating an animal (including human) patient with post-traumatic stress disorder caused or exacerbated by stress or anxiety, comprising administering to the patient an effective amount of dead cells of Lactobacillus fermentum and/or dead cells of Lactobacillus delbrueckii, including dead cells of Lactobacillus LB.

A further aspect provides a method of treating an animal (including human) patient with irritable bowel syndrome caused or exacerbated by stress or anxiety, comprising administering to the patient an effective amount of dead cells of Lactobacillus fermentum and/or dead cells of Lactobacillus delbrueckii, including dead cells of Lactobacillus LB.

A further aspect provides dead cells of Lactobacillus fermentum and/or dead cells of Lactobacillus delbrueckii, including dead cells of Lactobacillus LB, for use in the treatment of an animal (including human) patient with a condition with a behavioral, psychological and/or physical component caused or exacerbated by stress or anxiety.

A further aspect provides dead cells of Lactobacillus fermentum and/or dead cells of Lactobacillus delbrueckii, including dead cells of Lactobacillus LB, for use in the treatment of an animal (including human) patient with mood disturbances caused or exacerbated by stress or anxiety.

A further aspect provides dead cells of Lactobacillus fermentum and/or dead cells of Lactobacillus delbrueckii, including dead cells of Lactobacillus LB, for use in the treatment of an animal (including human) patient with autism or autism spectrum disorder (ASD) exacerbated by stress or anxiety.

A further aspect provides dead cells of Lactobacillus fermentum and/or dead cells of Lactobacillus delbrueckii, including dead cells of Lactobacillus LB, for use in a pharmaceutical composition for the treatment of an animal (including human) patient with stress or anxiety.

A further aspect provides dead cells of Lactobacillus fermentum and/or dead cells of Lactobacillus delbrueckii, including dead cells of Lactobacillus LB, for use in a pharmaceutical composition for the treatment of an animal (including human) patient with post-traumatic stress disorder caused or exacerbated by stress or anxiety.

A further aspect provides dead cells of Lactobacillus fermentum and/or dead cells of Lactobacillus delbrueckii, including dead cells of Lactobacillus LB, for use in a pharmaceutical composition for the treatment of an animal (including human) patient with a sociability disorder caused or exacerbated by stress or anxiety.

A further aspect provides dead cells of Lactobacillus fermentum and/or dead cells of Lactobacillus delbrueckii, including dead cells of Lactobacillus LB, for use in a pharmaceutical composition for the treatment of an animal (including human) patient with depression caused or exacerbated by stress or anxiety.

A further aspect provides dead cells of Lactobacillus fermentum and/or dead cells of Lactobacillus delbrueckii, including dead cells of Lactobacillus LB, for use in a pharmaceutical composition for the treatment of an animal (including human) patient with irritable bowel syndrome caused or exacerbated by stress or anxiety.

A further aspect provides a pharmaceutical composition comprising dead cells of Lactobacillus fermentum and/or dead cells of Lactobacillus delbrueckii, including dead cells of Lactobacillus LB, for use in the treatment of an animal (including human) patient with a condition with a behavioral, psychological and/or physical component caused or exacerbated by stress or anxiety

A further aspect provides a pharmaceutical composition comprising dead cells of Lactobacillus fermentum and/or dead cells of Lactobacillus delbrueckii, including dead cells of Lactobacillus LB, for use in the treatment of an animal (including human) patient with a mood disturbances caused or exacerbated by stress or anxiety.

A further aspect provides a pharmaceutical composition comprising dead cells of Lactobacillus fermentum and/or dead cells of Lactobacillus delbrueckii, including dead cells of Lactobacillus LB, for use in the treatment of an animal (including human) patient with autism or autism spectrum disorder (ASD) exacerbated by stress or anxiety.

A further aspect provides a pharmaceutical composition comprising dead cells of Lactobacillus fermentum and/or dead cells of Lactobacillus delbrueckii, including dead cells of Lactobacillus LB, for use in the treatment of an animal (including human) patient with stress or anxiety.

A further aspect provides a pharmaceutical composition comprising dead cells of Lactobacillus fermentum and/or dead cells of Lactobacillus delbrueckii, including dead cells of Lactobacillus LB, for use in the treatment of an animal (including human) patient with a sociability disorder caused or exacerbated by stress or anxiety.

A further aspect provides a pharmaceutical composition comprising dead cells of Lactobacillus fermentum and/or dead cells of Lactobacillus delbrueckii, including dead cells of Lactobacillus LB, for use in treating an animal (including human) patient with depression caused or exacerbated by stress or anxiety.

A further aspect provides a pharmaceutical composition comprising dead cells of Lactobacillus fermentum and/or dead cells of Lactobacillus delbrueckii, including dead cells of Lactobacillus LB, for use in treating an animal (including human) patient with post-traumatic stress disorder caused or exacerbated by stress or anxiety.

A further aspect provides a pharmaceutical composition comprising dead cells of Lactobacillus fermentum and/or dead cells of Lactobacillus delbrueckii, including dead cells of Lactobacillus LB, for use in treating an animal (including human) patient with irritable bowel syndrome caused or exacerbated by stress or anxiety.

A further aspect provides a pharmaceutical composition comprising dead cells of Lactobacillus fermentum and/or dead cells of Lactobacillus delbrueckii, including dead cells of Lactobacillus LB, for use in reducing corticosteroid levels in a human or non-human animal subject. Thus, for example, such dead cells can reduce cortisol levels in a human subject and corticosterone levels in a rodent such as a mouse.

In a first particular embodiment, the afore-mentioned dead cells of Lactobacillus fermentum and/or dead cells of Lactobacillus delbrueckii, including dead cells of Lactobacillus LB, in any of aspects 1 to 25 above are administered together with culture medium.

In a second particular embodiment, the afore-mentioned dead cells of Lactobacillus fermentum and/or dead cells of Lactobacillus delbrueckii, including dead cells of Lactobacillus LB, in any of aspects 1 to 25 above are administered as part of a culture solution, optionally also containing culture medium.

In a third particular embodiment, culture solution of particular embodiment 2 above is dried, e.g. freeze-dried, prior to administration.

In a fourth particular embodiment, the dried culture solution of particular embodiment 3 above is mixed with lactose prior to administration.

In a fifth particular embodiment, the product administered according to any of aspects 1 to 25 above is Lacteol®.

A further embodiment is a pharmaceutical composition comprising dead cells of Lactobacillus fermentum and/or dead cells of Lactobacillus delbrueckii, including dead cells of Lactobacillus LB, for use in treating a condition that can be improved by changing the composition and/or diversity of the human or non-human animal gut microbiota, for example by reducing in the gut the amount of Alistipes or Odoribacter species present.

Another embodiment is a pharmaceutical composition comprising dead cells of Lactobacillus fermentum and/or dead cells of Lactobacillus delbrueckii, including dead cells of Lactobacillus LB, for use in treating conditions with behavioral, psychological and/or physical components caused or exacerbated by stress or anxiety in an animal (including human) patient, by changing the composition and/or diversity of the human or non-human animal gut microbiota, for example by reducing the amount of Alistipes or Odoribacter species present in the gut.

### DESCRIPTION OF THE DRAWINGS

FIG. 1 is a schematic representation of a timeline for conducing the behavioral animal experiments.

FIG. 2 is a schematic representation of Open Field (OF) and Novel Object Recognition (NOR) tests, along with objects used for NOR test.

FIG. 3 shows the test cage before and after 30 minutes exploration during a Marble Burying (MB) experiment.

FIG. 4 is a schematic representation of the 3-Chambered Social Test (3CT).

FIG. 5 shows the apparatus used for the Elevated Plus Maze (EPM) test.

FIG. 6 shows the apparatus used for the Tail Suspension Test (TST).

FIG. 7 shows the average distance travelled (left graph), average time spend in the central zone (middle graph) and the speed of travel (right graph) for animals on Control (blue) or ADR-159 (orange) diets during the OF test. The error bars represent SEM. *p≤0.05; **p≤0.01

FIG. 8 shows the trace movements of representative animals on control (left trace) and ADR-159 (right trace) diets during the OF test.

FIG. 9 shows the average preference for novel objects in animals on Control (blue) or ADR-159 (orange) diets during the NOR test. Error bars represent SEM.

FIG. 10 shows the number of marbles buried by animals on Control (blue) or ADR-159 (orange) diets in the MB test. Error bars represent SEM.

FIG. 11 shows the trace movements of representative animals on Control (top trace) or ADR-159 (bottom trace) diets during habitation (left traces), sociability (middle traces) and social novelty (right traces) phases of the 3CT.

FIG. 12 shows the average time spend in individual chambers during habitation (A, B, C), sociability (D, E, F) and social novelty (G, H, I) phases by animals on Control (blue) or ADR-159 (orange) diets during the 3CT. Error bars represent SEM.

FIG. 13 shows the average speed (right graph) and distance travelled (left graph) for animals on Control (blue) or ADR-159 (orange) diets during the 3CT. Error bars represent SEM.

FIG. 14 shows the average time spend on interaction with either of the objects (total interaction time) (A, D, G) or individual objects (B, E, H) during habitation (A, B), sociability (D, E) and social novelty (G, H) phases by animals on control (blue) or ADR-159 (orange) diets during the 3CT. The discrimination ratio during the habitation, sociability and social novelty phases is presented in each of graphs (C), (F) and (I) respectively. Error bars represent SEM.

FIG. 15 shows the average time spend in closed (A) or open (D) arms, frequency to enter closed (B) or open (E) arms and latency to enter closed (C) or open (F) arms by animals on Control (blue) or ADR-159 (orange) diets during the EPM test. Error bars represent SEM.

FIG. 16 shows the average speed (left graph) and distance travelled (right graph) for animals on Control (blue) or ADR-159 (orange) diets during the EPM test. Error bars represent SEM.

FIG. 17 shows the average gut transition time in mice on Control (blue) or ADR-159 (orange) diets during the Carmine Red test. Error bars represent SEM.

FIG. 18 shows the average immobility time in animals on Control (blue) or ADR-159 (orange) diets during the TST. Error bars represent SEM. ***p≤0.001

FIG. 19 shows the average time of passive swimming for animals on Control (blue) or ADR-159 (orange) diets during the FST. Error bars represent SEM.

FIG. 20 shows the average base line (T0) corticosterone levels (left graph) and the change in corticosterone levels (right graph) before (T0) and 30, 60, 90 and 120 minutes after FST in animals on Control (blue) or ADR-159 (orange) diets. Error bars represent SEM. *p<0.05.

FIG. 21 shows the median relative abundance of major genera based on 16S rRNA sequences across time points in animals on control and ADR-159 diet. Color legend presented at the bottom. For simplicity, all genera with abundances below 1% were grouped together.

FIG. 22 shows PCoA plots of microbiota composition before (week 0) and during the diet intervention with Control (pink) and ADR-159 (blue) diet. Each dot represents an individual animal at each given time point. Ellipses represents the confidence interval of every group at 75%.

FIG. 23 shows selected differentially abundant OTUs within animals on control and ADR-159 diet. OTUs were selected based on criteria of differential abundance at a minimum of two of the final three time points (weeks 5, 6 and 8).

FIG. 24 shows a supplementary list of all the OTUs that were significantly different in animals on ADR-159 diet at any time point when compared to the microbiota of animals on control diet.

### DETAILED DESCRIPTION

The present disclosure relates to microbiological compositions having psycholeptic (calming) and psychoanaleptic (stimulating) effects, which are useful in protecting healthy subjects against the development of conditions with behavioral, psychological and/or physical components caused or exacerbated by stress or anxiety, and can positively impact existing conditions with behavioral, psychological and/or physical components caused or exacerbated by stress or anxiety.

The present disclosure also relates to the use of a composition containing dead cells of Lactobacillus fermentum, Lactobacillus delbrueckii or a mixture thereof, including Lactobacillus LB, to treat stress or anxiety, or treat or protect against conditions with behavioral, psychological and/or physical components caused or exacerbated by stress or anxiety, including, for example, depression, mood disturbances, conditions where sociability is dysfunctional, autism, autism spectrum disorder, post-traumatic stress disorder, chronic stress and a range of other stress-related diseases, and irritable bowel syndrome. Such compositions may also be useful to protect subjects against the effects of fibromyalgia, obsessive-compulsive behavior, or addictive behavior.

The Lactobacillus LB strain may conveniently be isolated from human feces and consists of two independent species, namely Lactobacillus fermentum and Lactobacillus delbrueckii. Lactobacillus LB in fermented culture medium is deposited at the Collection Nationale de Cultures de Microorganismes (CNCM) with the reference code MA 65/4E. Dead Lactobacillus LB cells may be obtained by heating the live cells in fermented culture medium at about 110° C. for about 1 hour. Dead cells of Lactobacillus fermentum, Lactobacillus delbrueckii or a mixture thereof may be obtained in a similar manner via a heat-killing process.

When used as a mixture, the weight ratio of Lactobacillus fermentum to Lactobacillus delbrueckii may be any suitable ratio from about 99:1 to about 1:99, e.g. about 9:1 to 1:9, including 9:1, 8:2, 7:3, 6:4, 5:5, 4:6, 3:7, 2:8, 1:9. The weight ratio of Lactobacillus fermentum to Lactobacillus delbrueckii may particularly be about 9:1.

Lacteol® may be prepared by drying dead cells of Lactobacillus LB together with the fermented culture medium (e.g. by lyophilization, spray-drying or fluid-bed drying) prior to formulating into a suitable composition for use in the present invention. In a particular aspect, lactose may be added to the wet fermented product prior to drying. In another aspect, lactose may also be added after drying as part of the formulation step.

Lacteol® and ADR-159 both contain a dried combination of heat-killed Lactobacillus fermentum and Lactobacillus delbrueckii in about a 9:1 ratio in culture medium. ADR-159 may be prepared in a similar manner to Lacteol®, except that ADR-159 is dried in a fluid bed rather than freeze-dried.

Dead cells of Lactobacillus fermentum, Lactobacillus delbrueckii or a mixture thereof, including Lactobacillus LB, may also be used in a liquid form, with or without lactose, by omitting the drying step or reconstituting the dried product with a suitable liquid such as water.

In representative murine behavioral tests, ADR-159 was incorporated into mice chow. Mice exposed continuously to ADR-159 over a prolonged period of time were subjected to a panel of murine behavioral tests. Similar behavioral tests were performed on control mice on an ADR-159 free diet.

The following behavioral tests were performed according to, or using procedures very closely related to, tests firmly established in the literature. However, slight variations and departures from the subsequently quoted literature procedures have been made in some tests, and therefore attention is drawn to the later Examples Section where the tests performed are described in detail.

### Murine Behavioral Tests

1. NOR: Cognitive function and memory were evaluated using the Novel Object Recognition (NOR) test—see Bevins R. A. et al., Nature Protocols, 2016 vol. 1 (3), pages 1306-1311.

2. OF and EPM: Anxiety-like behavior was evaluated using the Open Field (OF) and Elevated Plus Maze (EPM) tests—see Sweeney F F, O'Leary O F, Cryan J F, Activation but not blockade of GABAB receptors during early-life alters anxiety in adulthood in BALB/c mice. Neuropharmacology. 81:303-10 (2014).

3. 3CT: Anxiety-like behavior and sociability were evaluated in the Three-Chamber Test (3CT)—see Desbonnet L, Clarke G, Shanahan F, Dinan T G, Cryan J F, Microbiota is essential for social development in the mouse, Mol. Psychiatry. 19 (2):146-8 (2014).

4. MB: Quantification of behaviors relating to neophobia (fear of new things) was evaluated using the Marble Burying (MB) test—see Savignac H. M. et al., Official Journal of the European Gastrointestinal Motility Society, vol. 26 (11), pages 1615-1627 (2014). The MB test is also a measure of anxiety, compulsive behavior and stereotypical behavior.

5. TST: The Tail Suspension Test (TST) is widely used to screen compounds for potential antidepressant effects—see Steru L. et al., Psychopharmacology, vol. 85 (3), pages 367-370 (1985).

6. FST: The Forced Swim Test (FST) is also used to measure depressive behavior—see Porsoit R. D. et al., Archives Internationales de Pharmacodynamie et de Therapie, vol. 229 (2), pages 327-336 (1977) and Cryan J. F. et al., Molecular Psychiatry, vol. 9 (4), pages 326-357 (2004). It is a more stressful test than the TST, and therefore blood samples were taken before this test, and at regular intervals after this test, to measure corticosterone levels.

Unexpectedly, mice on an ADR-159 diet showed differences in behavior to mice on an ADR-159 free diet in a number of the aforementioned tests, including increased social interaction (3CT) and increased immobility (TST). Furthermore, physiological read-outs indicated lower base-line levels of corticosterone, a stress-related hormone, in mice on an ADR-159 diet relative to control mice. At the same time, the microbiota of mice on an ADR-159 diet underwent changes, suggesting the alteration of the microbiota composition as a potential mechanism for the behavioral changes seen.

The murine test results provide support for the administration of dead cells of Lactobacillus fermentum, Lactobacillus delbrueckii or a mixture thereof, including Lactobacillus LB, e.g. within a formulated product such as ADR-159 or Lacteol®, to animal (including human) patients to effectively manage stress and anxiety, enhance behavior patterns, and also protect against the development of, or treat, conditions caused or exacerbated by stress or anxiety.

In another aspect, a composition of the present disclosure may be useful in protecting against the development of a mood or social functioning disorder caused by alcohol and/or drug use, including amelioration of the use of a stress reliever such as smoking. In the case of drug use, the disorder may result from either abuse or the side effects of the drug at therapeutic doses.

In a further aspect, a composition of the present disclosure may be useful in treating stress and stress-related conditions resulting from withdrawal of a drug (e.g. nicotine) from a drug-addicted human subject.

The dead cells of Lactobacillus fermentum, Lactobacillus delbrueckii or a mixture thereof, including Lactobacillus LB, are present in a composition of the present invention in a sufficient amount to achieve the desired effect. In one exemplary embodiment of the invention, dead cells of the Lactobacillus LB strain are present in the proportion of about 1 billion or more cells/g, for example from about 10 to about 100 billion cells/g, including about 40 to about 80 billion cells/g (e.g. about 60 billion cells/g) in the composition of the present invention.

A composition of the present disclosure may be orally administered, and at a suitable dose, which will vary according to factors such as the subject's age, body weight and gender, the condition to be treated, and the duration of administration and the administration route. Ordinarily trained doctors or veterinarians can easily determine and prescribe an effective dose of a pharmaceutical composition of the present disclosure for the respective human or non-human animal patient. A pharmaceutical composition of the present disclosure in a suitable dosage form may be conveniently administered to the patient once or twice daily. In infants or younger children, based on a body weight ranging from 20 to 40 kg, approximately ½ of the adult dosage may be administered, and based on a body weight of less than 20 kg, approximately ¼ of the adult dosage may be administered.

A convenient unit dose of a composition of the present disclosure, e.g. in a standard pharmaceutical dosage form, such as a capsule or tablet, may be any effective dose up to about 2000 mg administered to an adult human patient once or twice daily.

A composition of the present disclosure may also be administered as a food or nutritional supplement or in a food, e.g. yoghurt. In this case very high doses up to about 100 g could be ingested.

A pharmaceutical composition of the present disclosure may be formulated using a pharmaceutically available carrier and/or excipient, and prepared in a unit capacity or contained in a high-dosage container according to a method that can be easily executed by one of ordinary skill in the art. Here, a dosage form may be a tablet, a capsule, a granule, powder, sachet containing powder, or liquids such as an aqueous medium-containing solution, a suspension, or an emulsion.

For example, to formulate a pharmaceutical composition as a capsule, dried (e.g. lyophilized) dead cells of Lactobacillus fermentum, Lactobacillus delbrueckii or a mixture thereof, including Lactobacillus LB, (optionally together with fermented culture medium and/or lyophilization additives) may be mixed with one or more suitable, non-toxic pharmaceutically available inactive carriers and excipients. Examples include binding agents, lubricants, disintegrating agents, diluents, coloring agents and desiccants. Suitable binding agent may be, but is not limited to, natural sugar such as starch, gelatin, glucose, or beta-lactose, a natural or synthetic gum such as corn sweetener, acacia, Tragacanth, or sodium oleate, sodium stearate, magnesium stearate, sodium benzoate, sodium acetate, or sodium chloride. The disintegrating agent includes, but is not limited to, starch, methylcellulose, agar, bentonite, or xanthan gum. Suitable lubricants include talc and magnesium stearate. Suitable desiccants include silicic acid and suitable diluents include a lactose such as anhydrous lactose. Suitable lyophilization additives include lactose monohydrate and a metal carbonate such as calcium carbonate. The product mixture may be contained in any standard capsule casing such as in a gelatin capsule.

In another example, a pharmaceutical composition of the present disclosure, in the form of a powder for an oral suspension, may be prepared by mixing dried (e.g. lyophilized) dead cells of Lactobacillus fermentum and/or Lactobacillus delbrueckii (optionally together with fermented culture medium and/or lyophilization additives) with one or more suitable, non-toxic pharmaceutically available inactive carriers and excipients. Examples include diluents, flavoring agents, sweetening agents and desiccants. Suitable desiccants include silicic acid and suitable diluents include a lactose such as anhydrous lactose or sucrose, the latter may also act as a sweetening agent. Suitable lyophilization additives include lactose monohydrate and a metal carbonate such as calcium carbonate. The powder product may be contained in any standard sachet ready for mixing with a drinkable liquid.

A composition for oral administration may also be part of a liquid or solid food or nutritional product (e.g. nutritional supplement). Examples include a milk, yoghurt or yoghurt-style product, a cheese, an ice-cream, a cereal-based product, a milk-based powder, a nutritional formula, an infant formula, a nutritional formula, a dried oral grit or powder, a wet oral paste or jelly, a grit or powder for dry tube feeding or a fluid for wet tube feeding.

Furthermore, optional additional active ingredients may also be present for use with a composition of the present disclosure. Optional active ingredients include, for example, vitamins, antibiotics, probiotics, prebiotics, anxiolytics, anti-depressants and mood-enhancing agents. The additional active ingredient(s) and a composition of the present disclosure may be co-administered or administered separately (e.g. sequentially) as individual compositions. Alternatively, the active ingredient(s) may be incorporated into the same composition as the dead cells of Lactobacillus fermenturn and/or Lactobacillus delbrueckii.

Suitable drug products for use in combination with a composition of the present disclosure include: selective serotonin reuptake inhibitors (SSRIs); serotonin-norepinephrine reuptake inhibitors; tricyclic anti-depressants; tetracyclic antidepressants; benzodiazepines; monoamine oxidase inhibitors; opioids and medications with opioid-like side effects.

While the present disclosure has been described herein with reference to certain exemplary embodiments and specific Examples, it will be understood by those skilled in the art that modifications in form and details may be made therein without departing from the spirit and scope of the invention.

### EXAMPLES SECTION

### A. Materials & Methods

### 1. Preparation of ADR-159-Supplemented Mouse Chow

An investigational variant of Lacteol® encoded ADR-159 was incorporated into standard mice chow [2018S Teklad Global 18% Protein Rodent Diet (Envigo)] to a final concentration of 5%.

### 2. Animals and Housing Conditions

24 eight-week old male C57BL/6 mice were used. Animals were randomly divided into six enriched (cardboard tubes and shredded paper) cages, each holding 4 mice, and allowed to acclimatize to their environment over 6 days. 12 animals (3 cages) were fed ad libitum throughout the whole study with mouse chow [2018S Teklad Global 18% Protein Rodent Diet (Envigo)] and the other 12 animals (3 cages) were fed ad libitum throughout the whole study with mouse chow supplemented with 5% ADR-159. The holding room was temperature (21±1° C.) and humidity (55±10%) controlled and under a 12-h light/dark cycle. All experiments were conducted in accordance with the European Directive 86/609/EEC, the Recommendation of 2007/526/65/EC and approved by the Animal Experimentation Ethics Committee of University College Cork. Animals were weighed each week and faecal samples were collected for 16S metagenomics. Additionally, feed consumption was monitored by weighing the feeders.

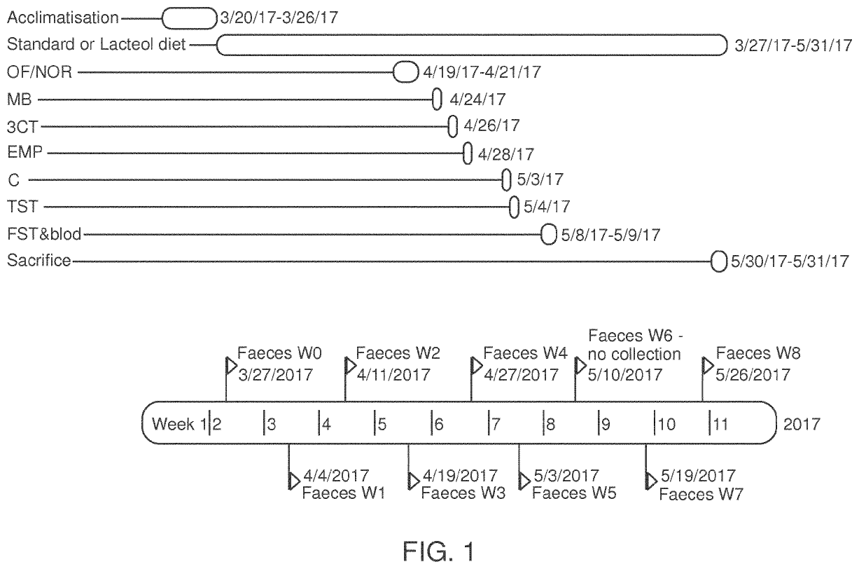

After 3 weeks of feeding the mice with chow or chow supplemented with 5% ADR-159, the animals were subjected sequentially to a battery of behavioral tests according to the protocol in FIG. 1. Individual animals (or cages of animals) allowed to acclimatize to the test room for 30-60 minutes prior the test, and were tested randomly to prevent bias. After a total of 8 weeks from initial feeding of the animals with chow or chow supplemented with 5% ADR-159, the mice were sacrificed and their trunk blood and whole brains (snap freeze on dry ice) collected.

### B. Behavioral Tests

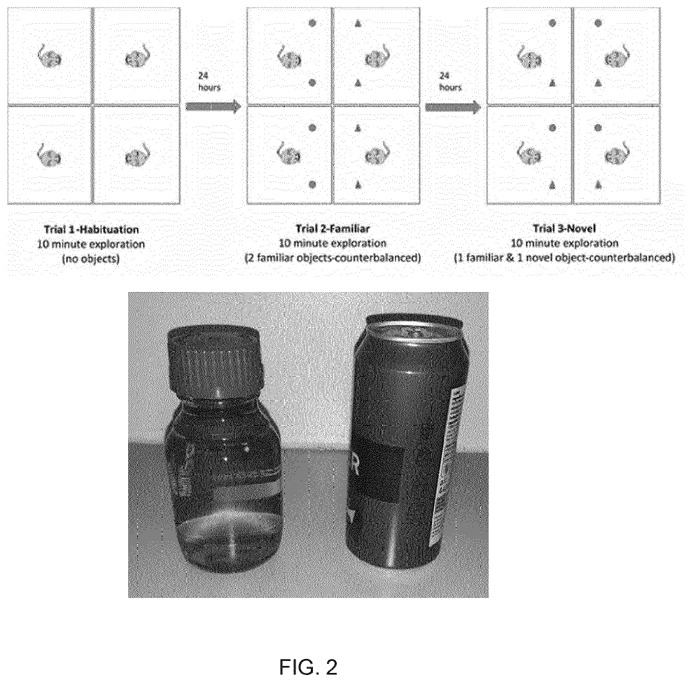

### 1. Open Field/Novel Object Recognition (OF/NOR)—See FIG. 2

A mouse were placed in the middle of a grey plastic rectangular box (40×32×23 cm, L×W×H) under a dim light (60 lux at the level of the arena) for 10 minutes to assess its response to a novel stressful environment and measure their locomotor activity. This is referred to as the “habitation phase”. 24 hours later, the same mouse was placed in the middle of the same box, this time with two identical objects (bottles or cans) for 10 minutes. This is referred to as the “familiar phase”. After another 24 h, the same mouse was placed in the same box for 10 minutes in which one of the two identical objects were substituted with a novel object (resulting in one bottle and one can in each box). This is referred to as the “novel phase”. The experiment was conducted in parallel with four different mice at a time. After each phase, the mice were returned to their home cages. The boxes and objects were cleaned with 70% alcohol to avoid any cue smell between each phase. Experiments were videotaped using a ceiling camera for further parameter analysis. Interaction with an object, include any contact with mouth, nose or paw, was scored using a stopwatch, and the percentage preference for a novel object over a familiar object was calculated. Climbing on top of the object was not considered an interaction. Additionally, Ethovision XT software v 8.5 (Noldus, TrackSys, Nottingham, UK) was used to measure the distance travelled within the box and time spend in the central zone (50% of the surface) during habitation phase.

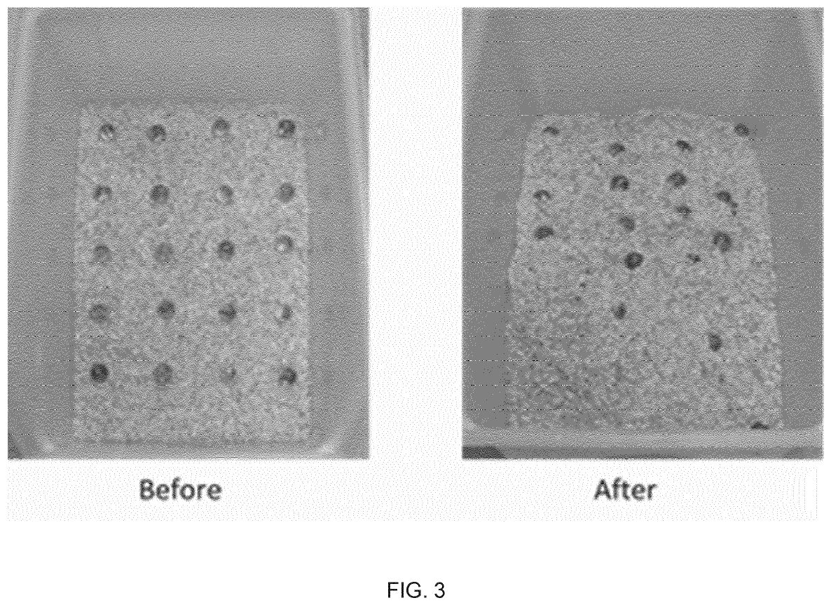

### 2. Marble Burying (MB)—see FIG. 3

Mice were individually placed in new boxes (38×25×18 cm, L×W×H) filled with sawdust (5 cm) and containing 20 marbles on the surface (five rows of marbles regularly spaced 2 cm away from the walls and 2 cm apart). After thirty minutes, the number of marbles where more than ⅔ of their surface was buried were noted. A higher number of marbles buried represents a higher level of anxiety. Boxes and marbles were cleaned with 70% alcohol between each experiment to avoid any cue smell.

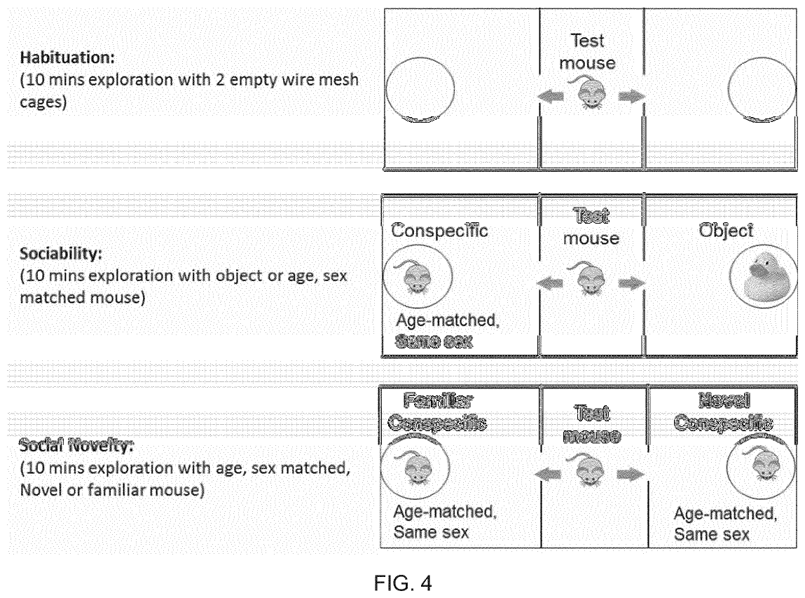

### 3. Three-Chambered Social Test (3CT)—see FIG. 4

### (a) Apparatus

The apparatus is a rectangle, three-chambered box (36 cm×19 cm). Dividing walls with semi-circular openings (3.5 cm high, 4.5 cm wide) allow access into each chamber. Two identical wire cup-like cages (bottom diameter 9 cm and 17 cm in height and bars spaced to allow contact but prevented fighting) are inside each side chamber in bilaterally symmetric positions.

### (b) Test

The test has three phases of 10 minutes each: 1) habitation 2) mouse versus object 3) novel mouse versus familiar mouse. Experiments were videotaped using a ceiling camera for further parameter analysis using two stopwatches. For the first phase the test mouse was placed into the middle chamber and allowed to explore the entire box (with empty small wire cages inside) for a 10 minute habituation session. After the habituation period, the test mouse was contained in the middle section by turning the partitions over for short interval while an object (yellow duck) is placed in a mesh cage in one side chamber and an unfamiliar conspecific male mouse (no prior contact with the test subject) in a mesh cage in other side chamber. During phase two, the partitions are turned around allowing mice to explore the entire box for 10 minutes. During the third phase, an object (yellow plastic duck) was replaced with an unfamiliar mouse serving as a novel mouse and in the other chamber the mouse used in phase two was kept the same, now serving as the familiar mouse. After every trial, all chambers and cup-like wire cages were cleaned with 70% ethanol and dried to prevent olfactory cue bias and to ensure proper disinfection. The amount of time spent exploring the object or mouse in each chamber was evaluated. The location of the unfamiliar mouse in the left vs right side chamber was systematically alternated between trials. Lack of innate side preference was confirmed during the initial 10 minutes of habituation to the entire arena. Time (in seconds) of interaction with each of the wire cages was measured and analyzed individually for each phase. The Discrimination Ratio (DR) was calculated for each phase as follows:

$$DR = \frac{( time of interaction with X )}{( time of interaction with X ) + ( time of interaction with Y )} * 100$$

The Discrimination Index (DI) was calculated for each of phases:

$$DI = \frac{( time of interaction with X ) - ( time of interaction with Y )}{( time of interaction with X ) + ( time of interaction with Y )} * 100$$

Where X and Y stand for:

| | | Mice vs. | New mice vs |
|---|---|---|---|
| | Habitation | Object | familiar mice |
| X | Empty | Mice | Novel mice |
| Y | Empty | Duck | Familiar mice |

Additionally, Ethovision XT software v 8.5 was used to measure time spend in each of the chambers as well as speed and distance travelled during each phase.

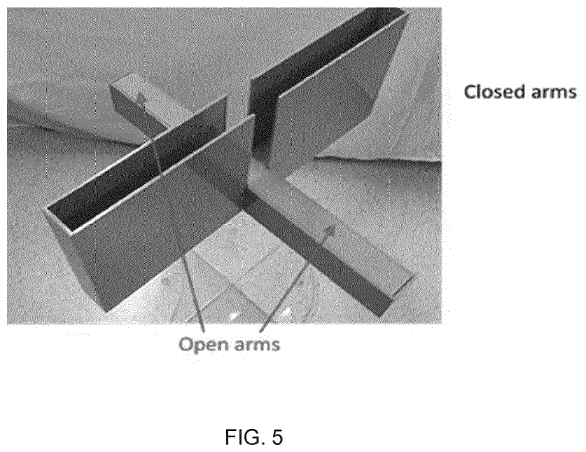

### 4. Elevated Plus Maze (EPM)—see FIG. 5

A grey plastic cross-shaped maze was elevated one meter from the floor, comprising two open (fearful) and two closed (safe) arms (arm length 30 cm; arm width 5 cm; wall high 20 cm or no wall). Experiments were conducted under red light (about 5 lux). Mice were individually placed into the center of the maze facing an open arm (to avoid direct entrance into a closed arm) and were allowed 5 minutes free exploration. The maze was cleaned with 70% alcohol to avoid any cue smell between each trial. Experiments were videotaped using a ceiling camera for further parameters analysis using Ethovision software (8.5 version, Noldus, TrackSys, Nottingham, UK). The time spent in an arm, the number of entries into an arm (entrance in an arm was defined as all four paws inside the arm) and latency (delay to enter) in each arm were measured along with velocity and distance moved.

### 5. Carmine Red (C)

Mice were kept individually without access to food or water for 3 hours, after which time the animals were gavaged with 100-200 μl of non-digestible Carmin red (6% solution in 0.5% methylcellulose). After gavage, cages with individual animals were monitored at 20 minute intervals until the first occurrence of red faecal pellet for each of the mice (maximum up to 7 h), after which time the mice was returned to the home cage. The transition time (minutes) was calculated as follows:

Transition time=(time of detection of 1st red pallet)−(time of gavage)

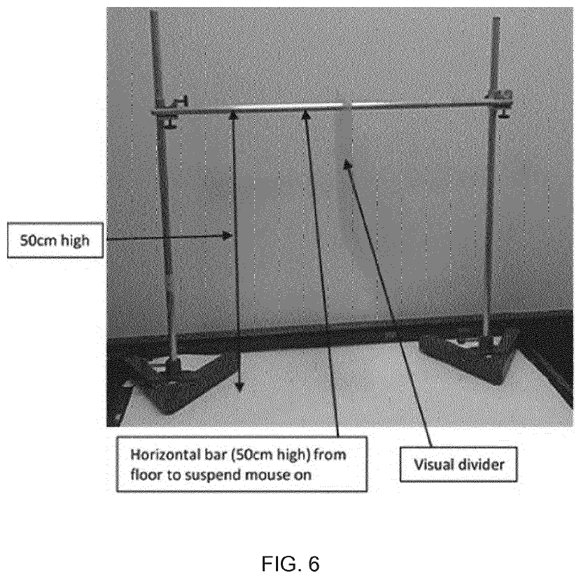

### 6. Tail Suspension Test (TST)—see FIG. 6

Mice were attached by the tail to a grid bar elevated 50 cm from the floor using adhesive tape (2 cm from tail tip). Two animals, separated by a visual divider, were subjected to the test for 6 minutes in parallel. Experiments were videotaped using a numeric tripod-fixed camera and data were further independently scored (Video Media Player software) by two experimenters blind to conditions. The time spent immobile was scored during the last four minutes of the test. Immobility is defined as the absence of voluntary or escape-orientated movement (grooming was considered mobility).

### 7. Forced Swim Test (FST)

Mice were individually placed in a clear glass cylinder (22cm diameter×45cm high), containing 15 cm depth water (23-25° C.). The water was changed between each test to remove odors. The test lasted 6 minutes and experiments were videotaped using a ceiling camera. Data were further scored using the videos (Video Media Player software) by two experimenters blind to conditions. The time of immobility as well as the time of passive swimming were scored over the last 4 minutes of the test. Immobility is defined as a total absence of movement, while passive swimming is defined as floating and movement without direction (e.g. motions to maintain the head above the water).

Blood samples for measurement of corticosterone levels were collected directly before the FST and 30, 60, 90 and 120 minutes after the FST from each animal. During the collection of blood, the mice were not restrained and the end of the tail was held with two fingers. Using a single edge razor blade a diagonal incision of 2-5 mm long was made at the end of the tail. Approximately 75 μl blood was collected in a heparinized glass capillary to avoid blood coagulation by increasing the pressure of the fingers on the tail above the incision. The collected blood was expelled into a collection tube and centrifuged at 3500×g at 4° C. temperature for 15 min. Plasma was carefully aspirated and stored at −80° C.

### C. Analysis

### 1. Corticosterone Assay

Samples were analyzed in duplicate in a single assay using corticosterone ELISA Kit (Enzo Life Scientific) according to manufacturer's recommendations. Steroid displacement reagent (SDR) was used to inhibit steroid binding to proteins. Plasma (15 μl) from base line samples (TO) was mixed with 15 μl of 1:100 SDR reagent and incubated, followed by addition of 270 μl of assay buffer, resulting in a 1:20 dilution. The remaining samples were diluted to a ratio of 1:40 by mixing 10 μl plasma with 10 μl of 1:100 SDR reagent and incubated, followed by addition of 380 μl of assay buffer. The threshold detection was less than 32 pg/ml (standard curve 32-20,000 pg/ml). Light absorbance was read with a multi-mode plate reader (Synergy 2, BioTek Instruments, Inc.) at 405 nm. The intensity of the bound yellow color is inversely proportional to the concentration of corticosterone in either standards or samples. The concentrations are expressed in ng/ml.

### 2. Statistical Analysis for Behavioral and Physiological Responses

Statistical analyses were conducted using SPSS software (IBM Corporation). Behavioral data above or below two standard deviations from the mean were classified as outliers and were removed prior to statistical analysis. Data that were normally distributed according to Shapiro-Wilk test were analyzed using parametric tests, an independent T-test. Behavioral non-parametric data were analyzed using the non-parametric Mann-Whitney U test or Wilcoxon Signed Rank test. Body weight, change in body weight and corticosterone data were analyzed using a repeated measure analysis of variances (ANOVA). Statistical significance was set at p<0.05.

### 3. 16S Metagenomics—Microbiota Analysis

### (a) DNA Isolation, Amplification, Indexing, Normalization and Sequencing

Faecal samples were stored at −80° C. until processing. DNA isolation was performed using QIAamp Fast DNA Stool mini kit (Qiagen) according to the manufacturer's recommendations, followed by measurement of DNA concentration using Qubit dsDNA HS Assay Kit and running 5 μl sample on a gel for quality assessment. V3 and V4 regions of 16S genes were amplified using Phusion Polymerase Master Mix and V3-V4 (Forward 5′-TCGTCGGCAGCGTCAGATGTGT ATAAGAGACAGCCTACGGGNGGCWGCAG-3′; Reverse 5′-GTCTCGTGGGCTCGGAG ATGTGTATAAGAGACAGGACTACHVGGGTATCTAATCC-3′) primers (98° C. 30 s; 25 cycles of 98° C. 10 s, 55° C. 15 s, 72° C. 20 s; 72° C. 5 min)—see Klindworth A, Pruesse E, Schweer T, Peplles J, Quast C, et al., Evaluation of general 16S ribosomal RNA gene PCR primers for classical and next—generation sequencing—based diversity studies. Nucleic Acids Res 41 (1) (2013). Amplicons were checked for quality and quantity by Qubit dsDNA HS Assay Kit and running on gel, and cleaned using Ampure XP magnetic beads. 5 μl of cleaned amplicon was used as a template for Index PCR using Phusion Polymerase Master Mix and Nextera XT Index Kit (95° C. 30 s; 8 cycles of 95° C. 30 s, 55° C. 30 s, 72° C. 30 s; 72° C. 5 min). Indexed amplicons were cleaned using Ampure XP magnetic beads and checked for quality and quantity by Qubit dsDNA HS Assay Kit and running on gel. All samples were normalized to 4 nM, followed by pooling together 5 μl of each sample and sending for MiSeq sequencing to GTAC (Germany).

### (b) Bioinformatic Analysis

First, Nextera adapters were removed using Cutadapt 1.9.1 [Martin M. EMBnetjournal, 2011 vol. 17, pages 10-22] as well as the first 5 bp and low-quality bases (PHRED quality lower or equal than 28) were trimmed using Trimmomatic [Bolger A M et al., Bioinformatics 2014 vol. 30 (15), pages 2114-2120]. Merging all paired FASTQ files, quality filtering by expected error rate, removal of singletons, and clustering sequences into operational taxonomic units (OTUs) was performed using UPARSE [Edgar R C. Nat Methods. 2013 vol. 10 (10), pages 996-998]. After that, a second chimera removal, based on the Ribosomal Database Project database (RDP v. 14; Cole J R et al. Nucleic Acids Res. 2014 vol 42 (database issue), D633-642) was performed using USEARCH v8.1 according to the UCHIME algorithm [Edgar R C et al. Bioinformatics 2011 vol. 27 (16), pages 2194-2200. Taxonomical classification of the 16S sequences was performed using the RDP classifier v. 2.12 [Wang Q et al. Applied and environmental microbiology 2007 vol. 73 (16), pages 5261-5267] against the RDP v 14, and SPINGO [Allard G et al. BMC bioinformatics 2015 vol. 16, page 324] for species level.

### (c) Statistical Tests

Several statistical tests were performed to analyze the microbial composition and diversity between control and treatment per week. First, to evaluate potential differences in the microbial composition between control and treatment per week, principal coordinates analyses (PCOA) plots using Bray-Curtis dissimilarity were constructed considering the number of sequences. Then, permutational multivariate analyses of variance (PERMANOVA; Anderson M J. Austral. Ecology 2001 vol. 26 (1), pages 32-46) were performed to assess differences in the microbial composition between control and treatment and per weeks. The maximum number of iterations was set to 1,000 in all analyses. As PERMANOVA does not discriminate between location and dispersion null hypotheses [Warton DI et al. Distance-based multivariate analyses confound location and dispersion effects. Methods Ecol Evol 3: 89-101 (2012)], generalised linear models for multivariate abundance data [Wang Y et al. mvabund: an R package for model-based analysis on multivariate data. Methods Ecol Evol 3: 471-474 (2012)] was performed as suggested by Warton et al. [Warton D I et al. Distance-based multivariate analyses confound location and dispersion effects. Methods Ecol Evol 3: 89-101 (2012)]. Second, to identify all species that were differentially expressed in the treatment respect to the control, two tests were performed: Analysis of Communities (ANCOM; Mandal S, et al. Microbial ecology in health and disease 2015 vol. 26, page 27663) was performed using the less stringent correction mode (equivalent to False Discovery Rate), and, differential expression analysis based on the Negative Binomial distribution (DESeq2; [Love M I et al. Moderated estimation of fold change and dispersion for RNA-seq data with DESeq2. Genome Biol 15: 550 (2014)] using Wald test. Third, to evaluate potential differences in the microbial diversity between control and treatment per week, nested ANOVA were performed when considering the Shannon and the inverse Simpson indexes. Shannon index was calculated using the decimal logarithm. Fourth, to describe the temporal trend of the microbial diversity over time, a Mann-Kendall trend test [Mann H B. Econometrica 1945 vol. 13 (3), pages 245-259] was performed. All these statistical approaches were carried out at an alpha level of 0.05, except DESeq2 analyses, which were carried out at an alpha level of 0.01, and were performed in R v. 3.4.1 using the ancom.R [Mandal S et al. Microbial ecology in health and disease 2015 vol. 26, page 27663], Kendall [Hipel K et al. Time Series Modelling of Water Resources and Environmental Systems, Elsevier 1994],), DESeq2 [Love M I et al. Moderated estimation of fold change and dispersion for RNA-seq data with DESeq2. Genome Biol 15: 550 (2014)], labdsv [Roberts D. labdsv: Ordination and Multivariate Analysis for Ecology 2016], mvabund [Wang Y et al. mvabund: an R package for model-based analysis on multivariate data. Methods Ecol Evol 3: 471-474 (2012)], and vegan [Oksanen J et al. vegan: community ecology package 2015] packages.

### D. Results

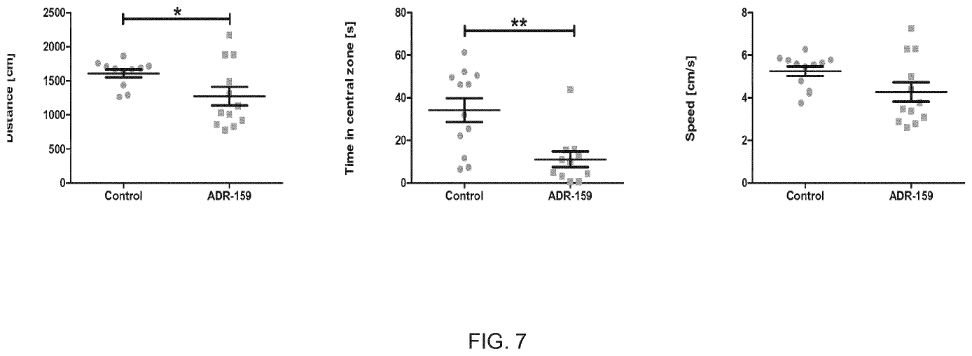

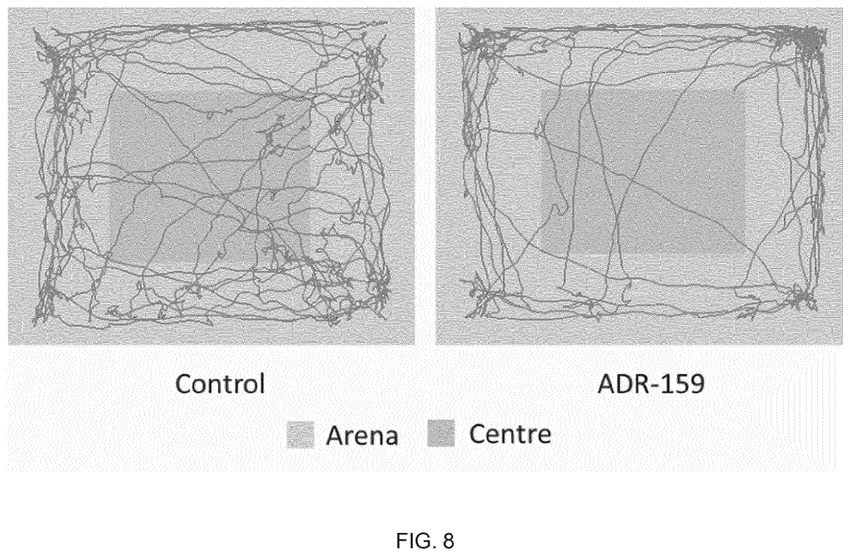

### 1. Open Field—See FIGS. 7 and 8

Time spend in the central zone was tested during the OF test for animals on control (n=12, Mean 34.23, SD=19.263) and ADR-159 (n=11, Mean 11.11, SD=12.155) diet. Data from the ADR-159 group were not normally distributed, and therefore a Mann-Whitney U test was used and the distribution of the time spend in central zone between diets was found to be statistically different (p=0.003).

Speed of travel was also tested during the OF test for animals on control (n=12, Mean 5.24, SD=0.787) and ADR-159 (n=12, Mean 4.27, SD=1.584) diet. Furthermore, distance travelled was tested during the OF test for animals on control (n=11, Mean 1606.92, SD=192.860) and ADR-159 (n=12, Mean 1272.34, SD=473.656) diet. An independent t-test was used for normally distributed data violating the condition of homogeneity of variances. This revealed that the mean distance difference of 334.58 cm (Control—ADR-159) was statistically significant (p=0.040, 95% CI of difference [17.516, 651.638]), whereas the mean difference in speed of travel of 0.98 cm/s (Control—ADR-159) was not statistically significant (p=0.073, 95% CI of difference [−0.103, 2.060]).

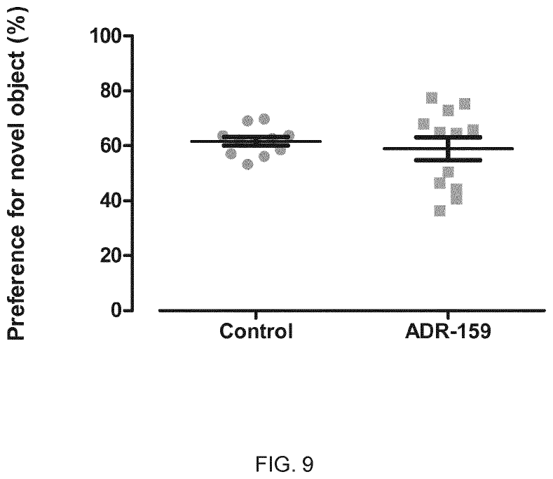

### 2. Novel Object Recognition—see FIG. 9

Preference for novel object was tested in animals on control (n=11, Mean 61.66, SD=5.100) and ADR-159 (n=12, Mean 58.95, SD=14.451) diet. Data from both groups were normally distributed and the condition of homogeneity of variances was violated. Hence, an independent t-test was used revealing that the mean difference of 2.70 (Control: ADR-159) was not statistically significant (p=0.553, 95% CI of difference [−6.841, 12.243]).

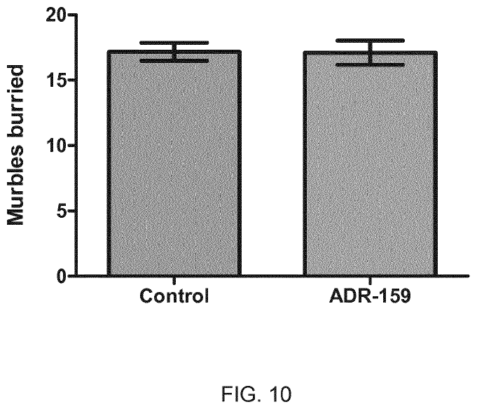

### 3. Marble Burying—See FIG. 10

The number of buried marbles was tested for 12 animals on control diet (Mean 17.17, SD=2.406) and 11 animals on ADR-159 diet (Mean 17.09, SD=3.113). Data from ADR-159 group were not normally distributed, hence a Mann-Whitney U test was used revealing that the distribution of number of buried marbles between diets was not statistically significant (p=0.833).

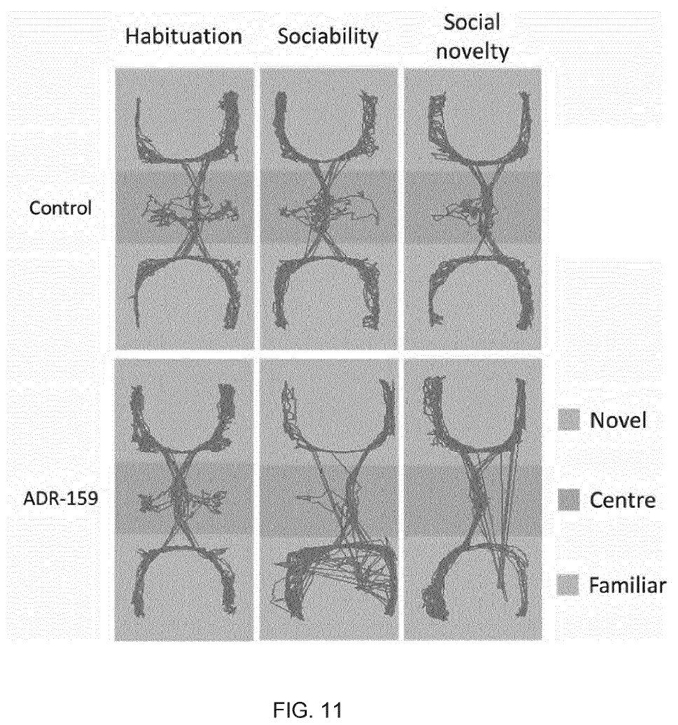

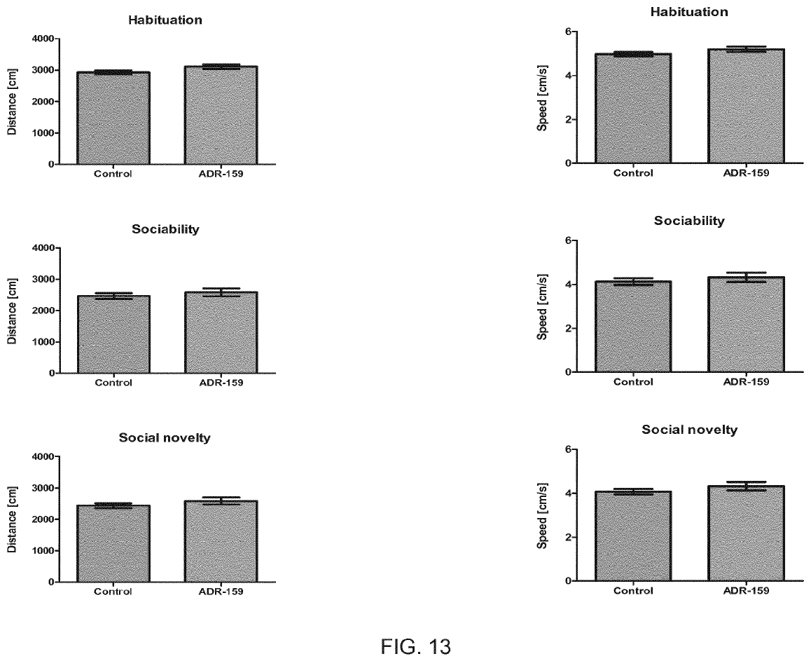

### 4. Three Chanber Test—see FIGS. 11 to 14

The impact of diet on time spent in the central chamber or in each of the side chambers with the object (empty basket or basket containing duck or mice) was tested in this study. Normally distributed data were analysed using independent T-test (when necessary with correction for violating equal variances assumption), while a Mann-Whitney U test was used for non-parametric data. Animal movements were traced during each of the experimental phases.

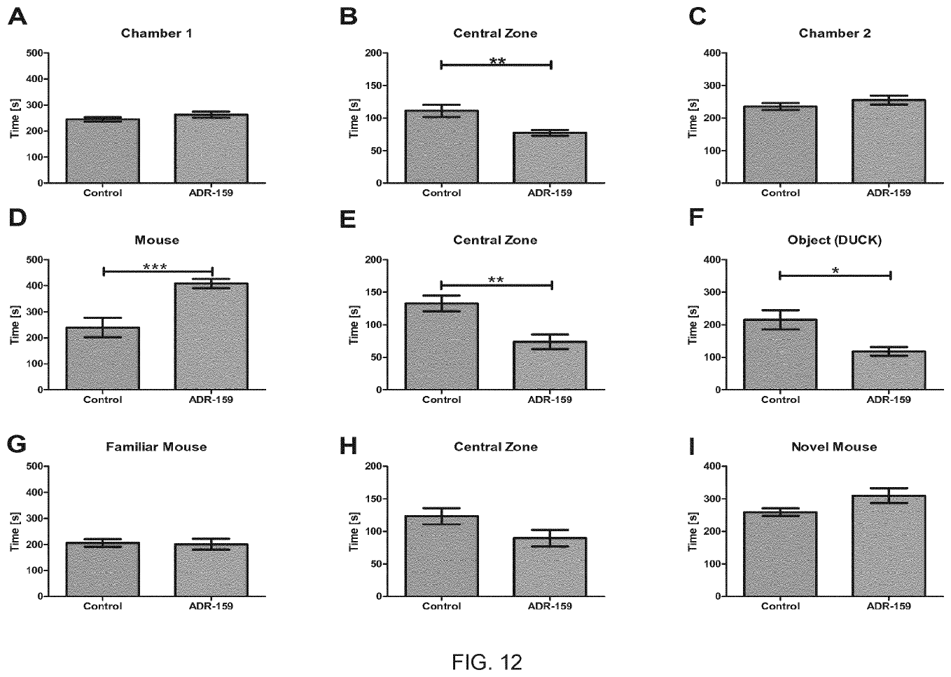

During the habitation (FIG. 12; top row) phase there was no significant difference in time spent in the empty chamber 1 (p=0.216) for animals on control (n=11, Mean 245.19, SD=29.550) or ADR-159 (n=12, Mean 263.43, SD=38.051) diet versus empty chamber 2 (p=0.268) (n=12, Mean 235.41, SD=38.297; n=12, Mean 255.06, SD=46.111, respectively). However, there was a significant difference (p=0.003) of 33.94 s in time spent in the central zone between animals on control (n=12, Mean 111.20, SD=32.389) or ADR-159 (n=11, Mean 77.26, SD=15.143) diet.

During the sociability (FIG. 12; middle row) phase there was a significant difference (p=0.001; mean difference (ADR-159-Control) 169.25 s; 95% CI of difference [82.65, 255.85]), in time spent in the chamber with mice for animals on control (n=12, Mean 238.20, SD=127.681) or ADR-159 (n=12, Mean 408.24, SD=60.516) diet. There was also a significant difference (p=0.002; mean difference (Control—ADR-159) 58.90 s; 95% CI of difference [24.79, 93.01]), in time spent in the central chamber for animals on control (n=11, Mean 132.62, SD=39.976) or ADR-159 (n=12, Mean 73.72, SD=38.666) diet. Finally, there was a significant difference (p=0.020; mean difference (Control—ADR-159) 96.97 s, in time spent in the chamber with the plastic duck for animals on control (n=12, Mean 215.04, SD=102.651) or ADR-159 (n=12, Mean 118.07, SD=46.472) diet.

During the social novelty (FIG. 12; bottom row) phase there was no significant difference in time spent in either of the chambers. In particular, in time spent in the chamber with the familiar mouse (p=0.837; mean difference (Control—ADR-159) 5.37; 95% CI of difference [−48.14, 58.89]) for animals on control (n=11, Mean 206.01, SD=48.405) and ADR-159 (n=12, Mean 200.64, SD=71.597) diet, or time spend in the central chamber (p=0.052) (n=12, Mean 123.36, SD=43.23; n=12, Mean 89.75, SD=43.70, respectively). Finally, there was no significant difference (p=0.067) in time spent in the chamber with the novel mouse between animals on control (n=12, Mean 259.27, SD=41.040) or ADR-159 (n=12, Mean 309.65, SD=79.135) diet.

Distance travelled and speed were tested in the 3CT test during habitation for animals on control (n=12, Mean 2931.74, SD=217.760 and n=11, Mean 4.97, SD=0.298, respectively) and ADR-159 (n=11, Mean 3106.77, SD=236.013 and n=11, Mean 5.20, SD=0.396, respectively) diet. During the sociability phase, distance and speed were as follows for animals on control (n=12, Mean 2467.41, SD=337.515 and n=12, Mean 4.13, SD=0.565, respectively) and ADR-159 (n=12, Mean 2585.44, SD=429.874 and n=12, Mean 4.32, SD=0.720, respectively) diet. Finally, during the social novelty phase tested parameters were for animals on control (n=11, Mean 2435.93, SD=253.744 and n=11, Mean 4.07, SD=0.424, respectively) and ADR-159 (n=11, Mean 2584.58, SD=373.289 and n=11, Mean 4.32, SD=0.627, respectively) diet. All data were normally distributed and the condition of homogeneity of variances was not violated. Hence, an independent t-test was used revealing that the mean differences in distance travelled and speed between animals on control and ADR-159 diet were not statistically significant (p>0.05).

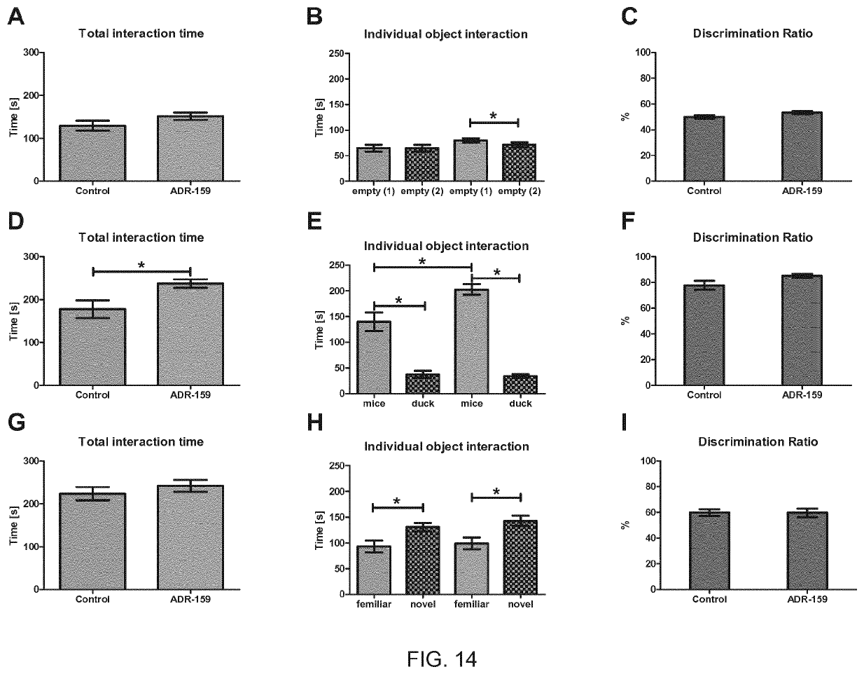

Additionally, the impact of diet on direct interaction with objects (empty basket or basket containing duck or mice) was tested in this study. The total interaction time with either of the objects were tested during habitation, sociability and social novelty phases for 12 subjects on control diet (Mean 129.33, SD=39.246; Mean 177.83, SD=71.730; and Mean 223.42, SD=54.755, respectively) and 12 subjects on ADR-159diet (Mean 151.58, SD=29.432; Mean 237.50, SD=34.503; and Mean 241.75, SD=47.858, respectively) (FIG. 14, graphs A, D and G). All data were normally distributed, and the condition of homogeneity of variances was not violated for habitation and social novelty phase. Hence, an independent t-test was used, revealing that the mean difference in interaction time during sociability phase of 59.67 s (p=0.020, 95% CI of difference [10.91, 108.42]) was statistically significant. However, the mean difference in interaction time during habitation and social novelty phase of 22.25 s (p=0.130, 95% CI of difference [−7.12, 51.62]) and 18.33 s (p=0.392, 95% CI of difference [−25.20, 61.87]), respectively, were not statistically significant.

Discrimination ratio between two objects was analyzed during habitation, sociability and social novelty phase for animals on control diet (n=11, Mean 49.93, SD=4.579; n=12, Mean 77.71, SD=12.525; and n=11, Mean 59.81, SD=9.224, respectively) and 12 subjects on ADR-159 diet (Mean 53.29, SD=4.341; Mean 85.10, SD=6.028; and Mean 59.81, SD=9.224, respectively)

(FIG. 14, graphs C, F and I). Data for habitation and social novelty phases were normally distributed, and the condition of homogeneity of variances was not violated. Hence, an independent t-test was used revealing that the mean difference in discrimination ratio during habitation and social novelty phases of 3.36 (p=0.086, 95% CI of difference [−0.511, 7.225]) and 0.21 (p=0.963, 95% CI of difference [−9.166, 9.592]), respectively, were not statistically significant. Data for sociability were not normally distributed, hence, a Mann-Whitney U test was used revealing that the distribution of discrimination ratios between diets was not statistically different (p=0.114).

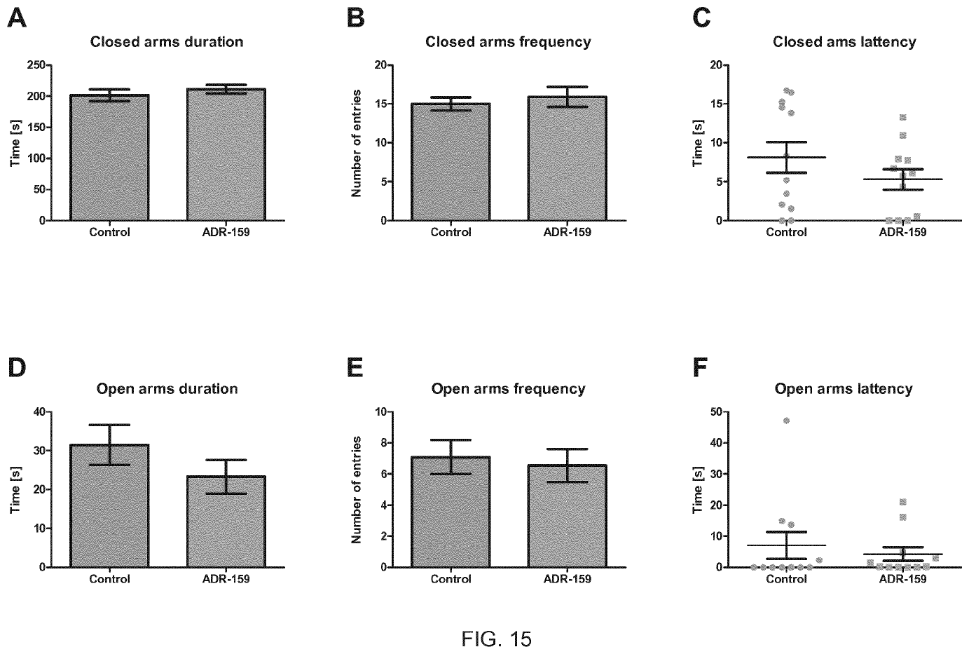

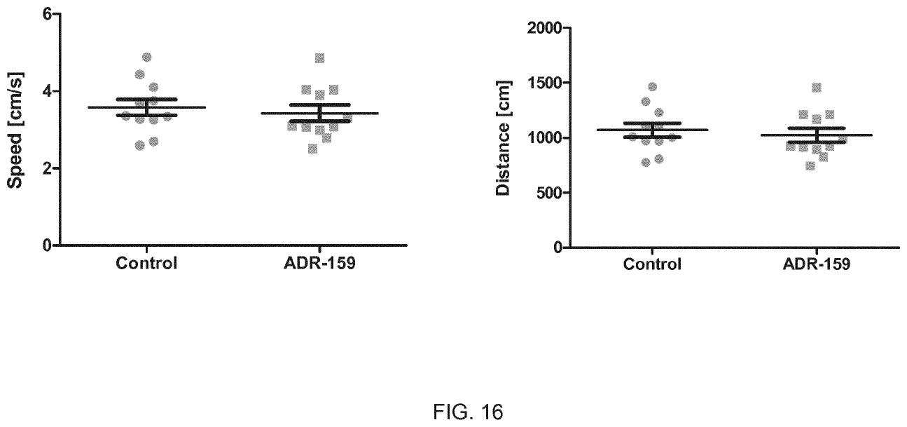

### 5. Elevated Plus Maze—See FIGS. 15 and 16

ADR-159 diet versus control diet was tested for up to 12 animals per group. Normally distributed data not violating equal variances assumption were analysed using an independent T-test, while a Mann-Whitney U test was used for non-parametric data. The difference in time spent in either closed (p=0.416) or open arms (p=0.237), frequency of entering (p=0.695; p=0.724) and latency to enter (p=0.319; p=0.519) were not statistically significant.

Speed and distance travelled were tested during the EPM for 11 animals on control diet (Mean 3.58, SD=0.691 and Mean 1069.57, SD=208.827, respectively) and 11 animals on ADR-159 diet (Mean 3.42, SD=0.695 and Mean 1023.61, SD=211.157, respectively). All data were normally distributed and the condition of homogeneity of variances was not violated. Hence, an independent t-test was used revealing that the mean speed difference of 0.15 cm/s (Control—ADR-159) was not statistically significant (p=0.607, 95% CI of difference [−0.46, 0.77]). Also, the mean difference in distance travelled of 45.96 cm (Control—ADR-159) was not statistically significant (p=0.613, 95% CI of difference [−140.83, 232.74]).

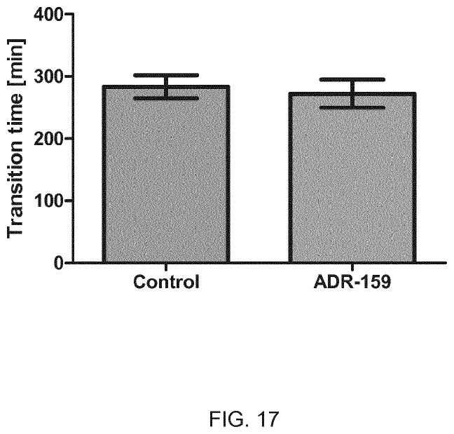

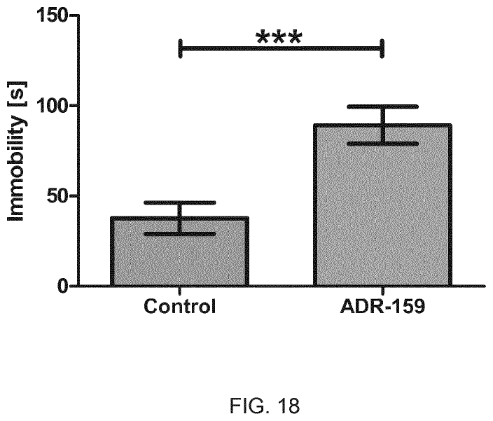

### 6. Carmine Red—See FIG. 17

The impact of diet on the gut transition time was tested. Data from the ADR-159 group were not normally distributed, hence a Mann-Whitney U test was used and the distribution of transition times between animals on control (n=11, Mean 283.00, SD=62.495) and ADR-159 (n=12, Mean 272.18, SD=74.737) diets was found to be statistically the same (p=0.748).

### 7. Tail Suspension Test—See FIG. 18

The impact of diet on the time of immobility during the TST was tested for 11 animals on control diet (Mean 37.63, SD=28.608) and 11 animals on ADR-159 diet (Mean 89.18, SD=33.828) (FIG. 18). Data from both groups were normally distributed and the condition of homogeneity of variances was not violated. Hence, an independent t-test was used and revealed that the mean difference of 51.55 (ADR-159-Control) was statistically significant (p=0.001, 95% CI of difference [23.682, 79.410]).

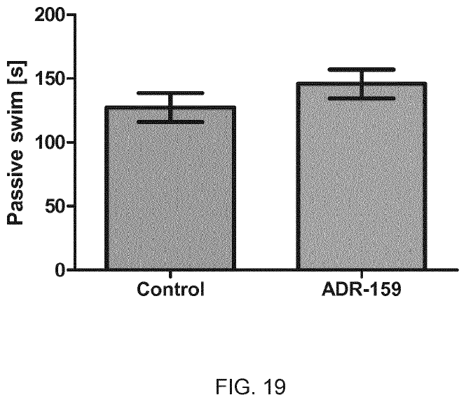

### 8. Forced Swim Test—See FIG. 19

The impact of diet on the time of passive swimming during FST was tested for animals on a control (n=11, Mean 118.86, SD=45.994) and ADR-159 (n=11, Mean 136.57, SD=47.607) diet. Data from both groups were normally distributed and the condition of homogeneity of variances was not violated. Hence, an independent t-test was used and the mean difference of 17.71 (ADR-159-Control) was not statistically significant (p=0.364, 95% CI of difference [−21.92, 57.34]).

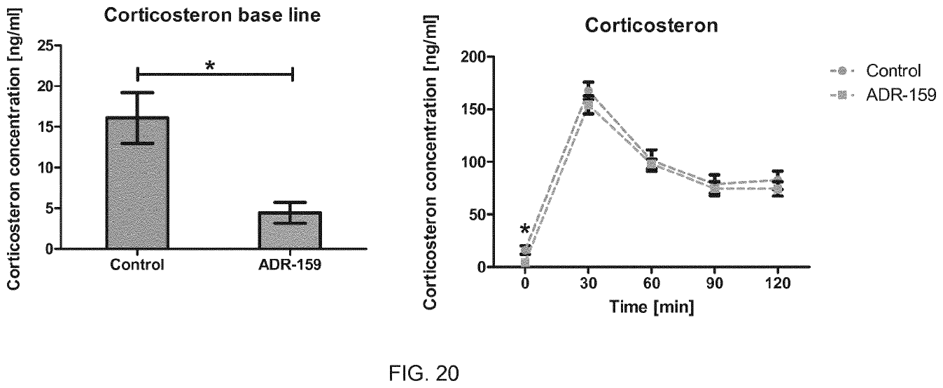

### 9. Corticosterone Levels—See FIG. 20

The base line corticosterone levels was tested before the FST in animals on control (n=11, Mean 16.10, SD=13.855) and ADR-159 (n=10, Mean 4.33, SD=4.469) diet. Data from both groups were normally distributed and the condition of homogeneity of variances was violated. Hence, an independent t-test was used showing that the mean difference of 11.77 (Control—ADR-159) was statistically significant (p=0.020, 95% CI of difference [2.18, 21.36]).

Following the FST, the impact of diet on the change of corticosterone levels in time was tested. Repeated measure ANOVA for 11 subjects on control diet and 10 subjects on ADR-159 diet revealed no statistically significant differences p=0.230 F=1.537 between treatments.

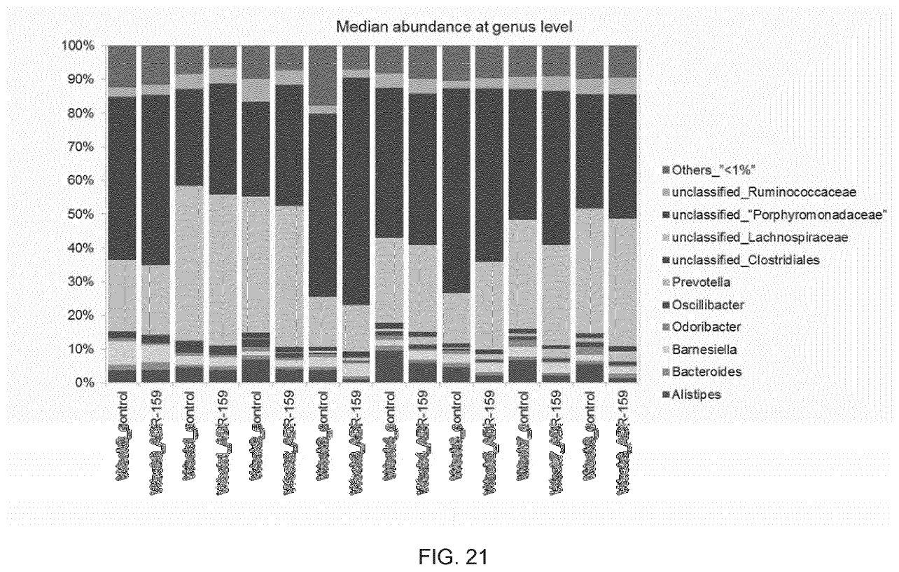

### 10. Microbiota Analysis—See FIGS. 21 to 24

The impact of diet on the murine microbiota was tested for animals on control and ADR-159 diet. Firmicutes, Bacteroidetes and Verrucomicrobia were the three most abundantly represented phyla in faeces of animals on both control and ADR-159 diets. At a genus level the microbiota of all animals was dominated by unclassified Porphyromonadaceae and unclassified Lachnospiraceae (FIG. 21). Overall, the microbial composition at the genus level was comparable between animals on control and ADR-159 diet. The median relative abundance of Alistipes and Odoribacter was consistently reduced in animals on ADR-159, while Prevotella was increased compared to animals on control diet.

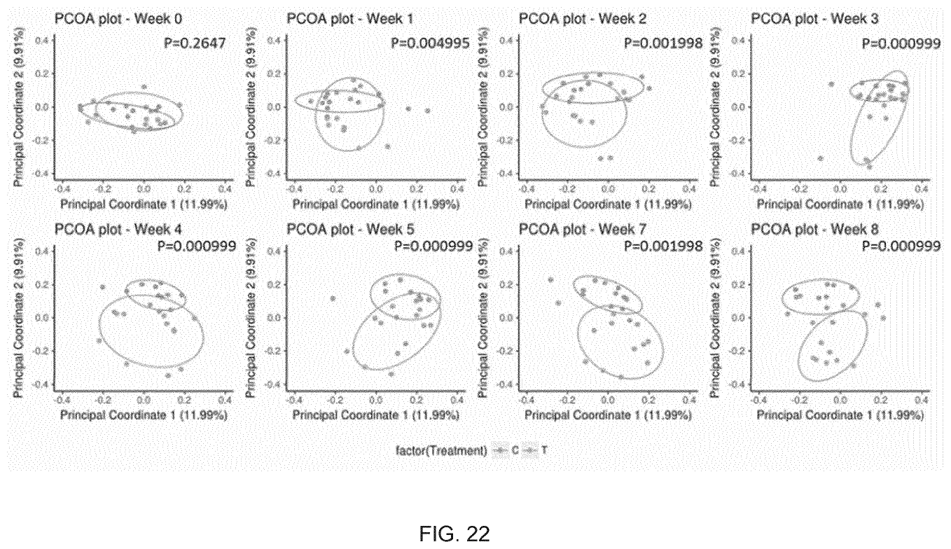

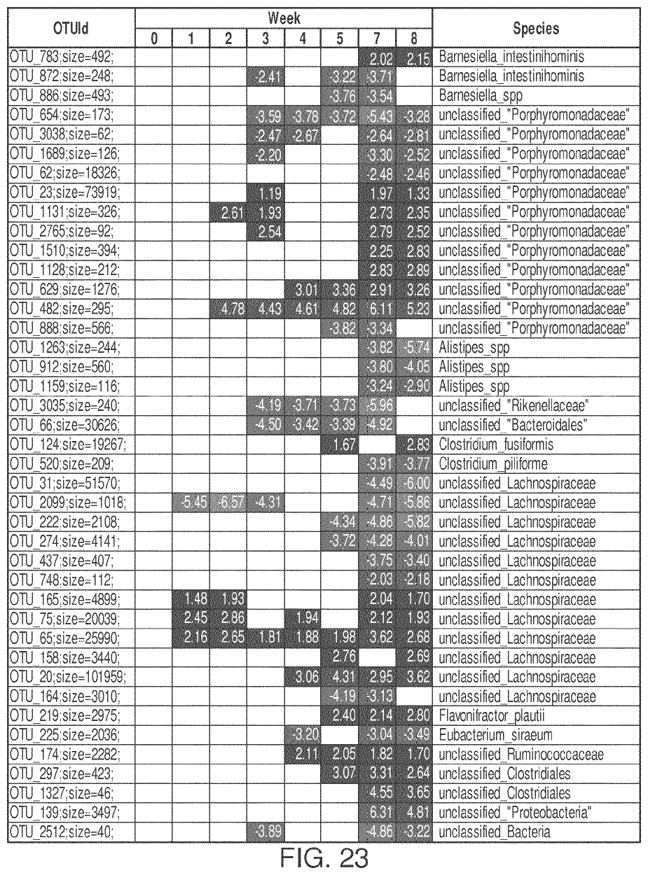

Microbiota diversity between individuals was visualised by means of principal coordinate analysis (PCoA) plots and analysed by PERMANOVA. PCoA plots revealed a clear time-related separation of microbiota of animals on control and ADR-159 diets (FIG. 22). While prior to the diet differentiation the microbiota of all animals were comparable and closely clustered, after eight weeks of diet microbiota of animals on ADR-159 diet clearly separated from the control microbiota.

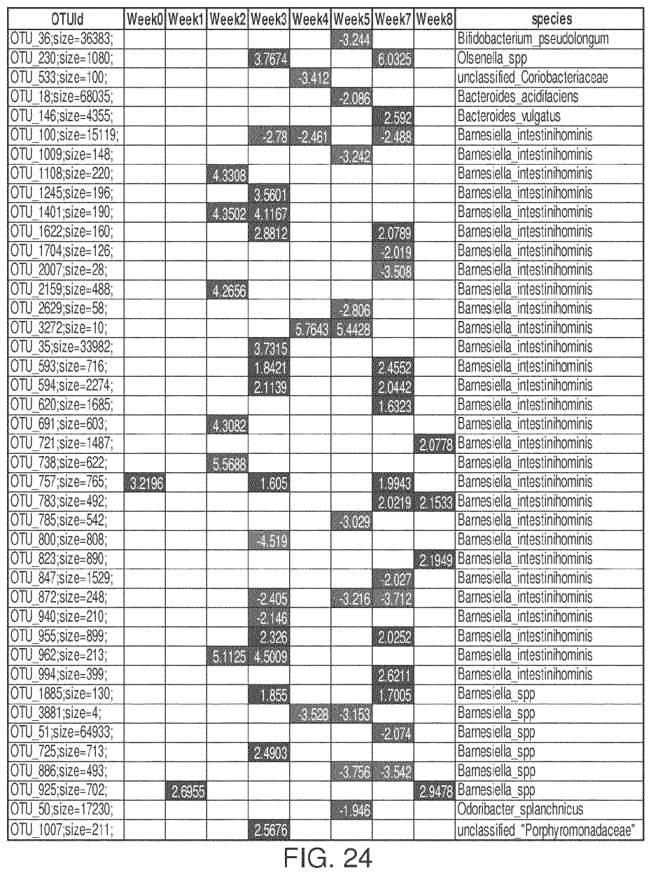

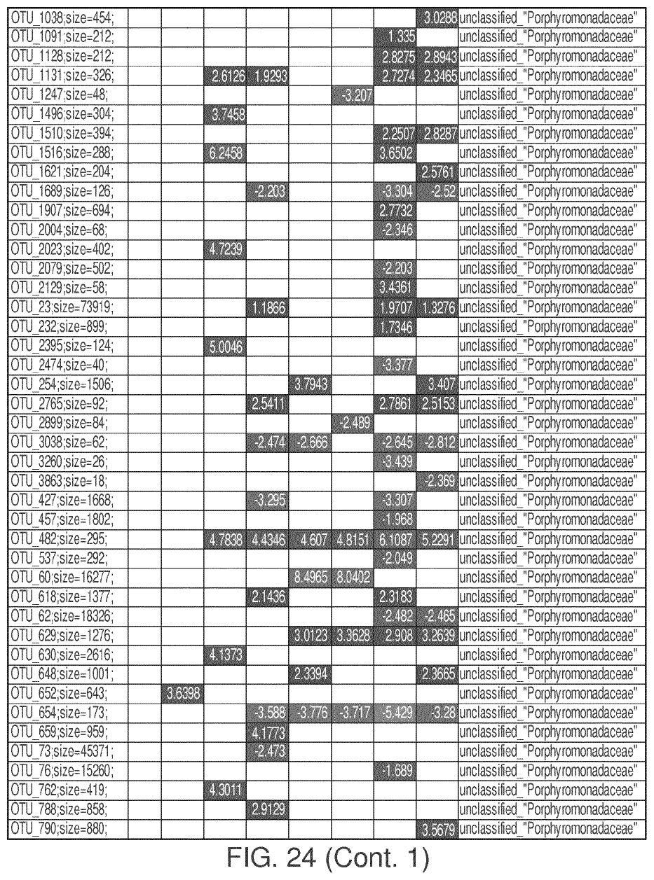

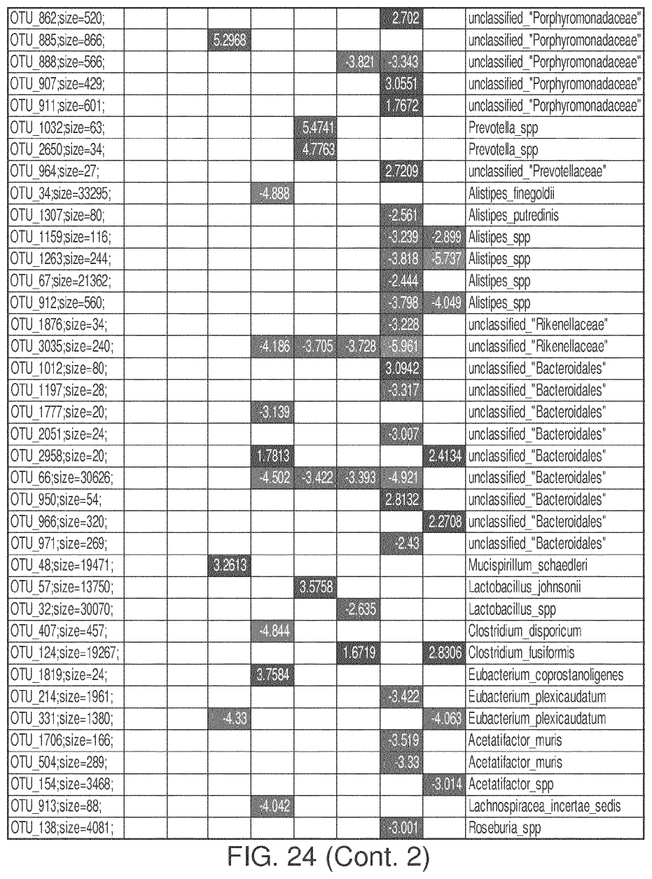

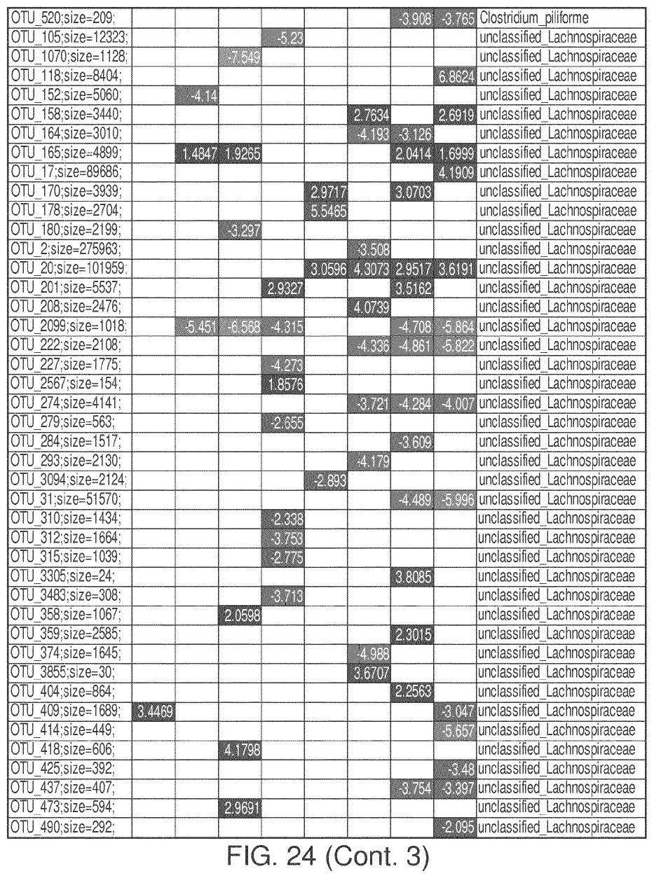

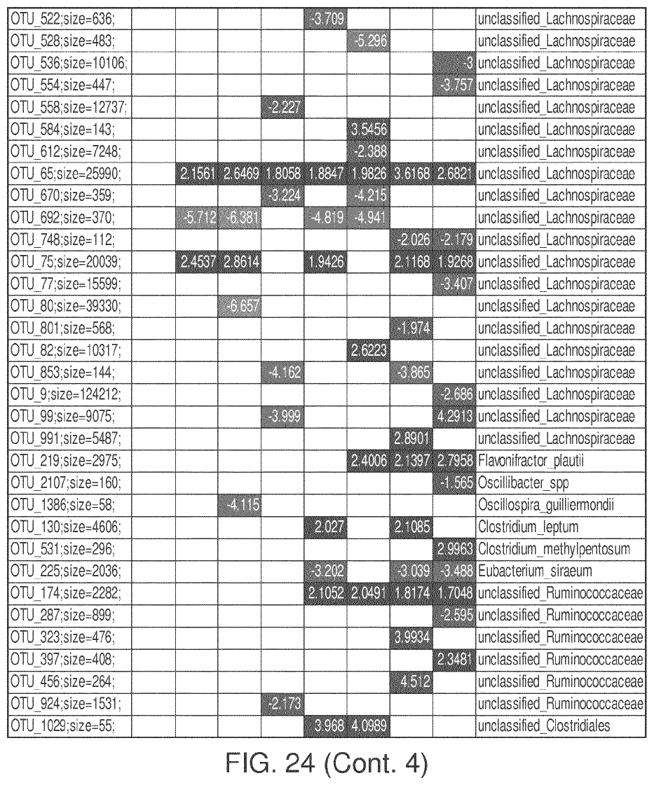

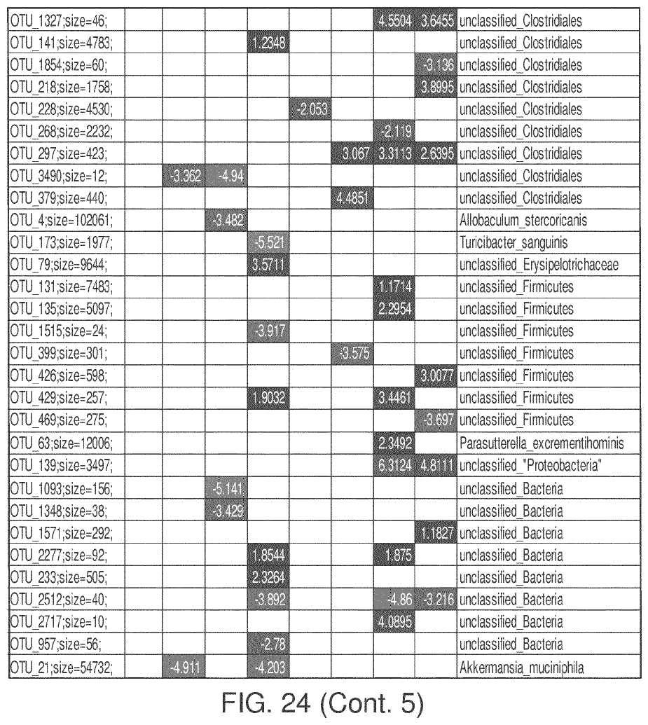

Next, changes in relative abundance between animals on control and ADR-159 diet were analysed at the OTU level (FIG. 23, 24), with 229 OTUs differently abundant in at least one time point (FIG. 24. To focus on the long term effect of the dietary intervention we looked particularly at 41 OTUs showing abundance changes in the period from 5 to 8 weeks of the experiment (only including those OUT' s which showed changes in at least two of the final three time points) (FIG. 23). Interestingly, some of the OTUs belonging to unclassified Porphyromonadaceae and unclassified Lachnospiraceae showed increased abundance in ADR-159 animal while other OTUs from the same taxa showed reduced abundance.

### E. Discussion

Compositional 16S sequencing confirms that the inclusion of ADR-159 in the mouse diet has a significant effect both on the composition of the microbiota and on aspects of animal behaviour. We believe this to be the first study to demonstrate such a simultaneous effect. The results provide a potentially novel mechanistic insight on the impact of heat inactivated bacteria and their metabolites on the gut-brain axis.

1. A composition comprising dead cells of Lactobacillus fermentum and/or dead cells of Lactobacillus delbrueckii for use to produce a psychobiotic effect in a human or non-human animal subject.

2. A composition according to claim 1, wherein said psychobiotic effect is achieved by changing the composition and/or diversity of the human or non-human animal gut microbiota.

3. A composition according to claim 1 or claim 2, wherein said psychobiotic effect is achieved by modifying (e.g. reducing) the amount of Alistipes and/or Odoribacter species present in the human or non-human animal gut.

4. A composition according to any one of claims 1 to 3, wherein said psychobiotic effect protects a human or non-human animal subject against the development of a condition with a behavioral, psychological and/or physical component.

5. A composition comprising dead cells of Lactobacillus fermentum and/or dead cells of Lactobacillus delbrueckii for use in protecting a human or non-human animal subject against the development of a condition with a behavioral, psychological and/or physical component caused or exacerbated by stress or anxiety.

6. A composition according to claim 5 for use in protecting a human or non-human animal subject against the development of a condition selected from anxiety, depression, mood disturbances, a condition where sociability is dysfunctional, autism, autism spectrum disorder, irritable bowel syndrome, post-traumatic stress disorder, chronic stress and a range of other stress-related diseases.

7. A composition for use according to any one of claims 1 to 6, wherein the subject is a healthy human or healthy non-human animal.

8. A composition comprising an effective amount of dead cells of Lactobacillus fermentum and/or dead cells of Lactobacillus delbrueckii for use in treating anxiety, stress or a stress-related condition in a human or non-human animal subject.

9. A composition for use according to claim 8, wherein said stress or stress-related condition is a consequence of withdrawing a drug (e.g. nicotine) from a drug-addicted human subject.

10. A composition comprising dead cells of Lactobacillus fermentum and/or dead cells of Lactobacillus delbrueckii for use in reducing corticosteroid levels in a non-human animal subject.

11. A composition comprising dead cells of Lactobacillus fermentum and/or dead cells of Lactobacillus delbrueckii for use in reducing corticosteroid levels in a human subject.

12. A composition for use according to any one of claims 1 to 11, comprising a mixture of dead cells of Lactobacillus fermentum, dead cells of Lactobacillus delbrueckii and culture medium.

13. A composition for use according to claim 12, wherein said dead cells of Lactobacillus fermentum and dead cells of Lactobacillus delbrueckii are present in the mixture at a weight ratio of about 9:1.

14. A composition for use according to claim 12 or claim 13, wherein the said mixture is dried (e.g. lyophilized).

15. A composition for use according to any one of claims 1 to 14, comprising an effective amount of Lacteol®.

16. A composition for use according to any one of claims 1 to 15, wherein the composition is in the form of a pharmaceutical composition, a food supplement, or a nutritional supplement.

17. A composition for use according to claim 16, wherein said food supplement or nutritional supplement is comprised within a food product selected from milk, yoghurt or yoghurt-style product, cheese, ice-cream, a cereal-based product, a milk-based powder, an infant formula, a nutritional formula, a dried oral grit or powder, a wet oral paste or jelly, a grit or powder for dry tube feeding or a fluid for wet tube feeding.

18. A pharmaceutical composition for use according to any one of claims 1 to 17, comprising one or more excipients (e.g. lactose).

19. A method of protecting a human or non-human animal subject against the development of a condition with a behavioral, psychological and/or physical component caused or exacerbated by stress or anxiety, comprising administering to the subject an effective amount of dead cells of Lactobacillus fermentum and/or dead cells of Lactobacillus delbrueckii.

20. A method according to claim 19 wherein said dead cells of Lactobacillus fermentum and/or dead cells of Lactobacillus delbrueckii are administered in an amount sufficient to produce a psycholeptic or psychoanaleptic effect.

21. A method according to claim 19 or claim 20, wherein said condition is selected from anxiety, depression, mood disturbances, a condition where sociability is dysfunctional, autism, autism spectrum disorder, irritable bowel syndrome, post-traumatic stress disorder, chronic stress and a range of other stress-related diseases.

22. A method according to any one of claims 19 to 21, wherein the subject is a healthy human or healthy non-human animal.

23. A method of treating anxiety, stress or a stress-related condition in a human or non-human animal subject, comprising administering to the subject an effective amount of dead cells of Lactobacillus fermentum and/or dead cells of Lactobacillus delbrueckii.

24. A method according to claim 23, wherein said stress or stress-related condition is a consequence of withdrawing a drug (e.g. nicotine) from a drug-addicted human subject.

25. A method according to any one of claims 19 to 24, comprising administering a mixture of dead cells of Lactobacillus fermentum, dead cells of Lactobacillus delbrueckii, and culture medium.

26. A method according to claim 25, wherein said dead cells of Lactobacillus fermentum and dead cells of Lactobacillus delbrueckii are present in the mixture at a weight ratio of about 9:1.

27. A method according to claim 25 or claim 26, wherein said mixture is dried (e.g. lyophilized).

28. A method according to any one of claims 19 to 27, comprising administering an effective amount of Lacteol®.
